(19) United States
(12) Patent Application Publication
(10) Pub. No.: US 2005/0118171 A1
(43) Pub. Date: Jun. 2, 2005

(54) Treatment of rheumatoid arthritis with CD99 antagonists

(75) Inventors: Vincent Jacques Hurez, Albany, CA (US); Leif Gustaf Wennerberg, Mountain View, CA (US); Didier A. Scherrer, Mountain View, CA (US); Seth G. Michelson, San Jose, CA (US)

Correspondence Address: FISH & RICHARDSON P.C., 3300 DAIN RAUSCHER PLAZA, MINNEAPOLIS, MN, 55402, US

(73) Assignee: Entelos, Inc., Foster City, CA (US)

(21) Appl. No.: 10/938,134

(22) Filed: Sep. 10, 2004

Related U.S. Application Data
(60) Provisional application No. 60/502,345, filed on Sep. 11, 2003.

(51) Int. Cl. A61K 39/395; G01N 33/53

(52) U.S. Cl. 424/144.1; 435/7.1

(57) ABSTRACT

The invention encompasses novel methods of treating rheumatoid arthritis and its symptoms and novel methods of identifying and screening for drugs useful in the treatment of rheumatoid arthritis and its clinical symptoms. Targeted manipulation of a computer model of a human rheumatic joint provided the surprising result that the activity of CD99, an adhesion molecule known to have an effect on some cancers, has a significant impact on the pathophysiology of rheumatoid arthritis. Inhibition of the activity of CD99 is predicted to alleviate the symptoms of rheumatoid arthritis.

### A. RELATED APPLICATIONS

This application claims the benefit of U.S. Provisional Application No. 60/502,345, filed Sep. 11, 2003 which is herein incorporated by reference.

### B. FIELD OF THE INVENTION

This invention relates to novel methods of treating rheumatoid arthritis and methods of identifying compounds useful in treating rheumatoid arthritis.

### C. BACKGROUND OF THE INVENTION

There are more than 100 forms of arthritis and of them, rheumatoid arthritis is the most painful and crippling form. Rheumatoid arthritis, a common disease of the joints, is an autoimmune disease that affects over 2 million Americans, with a significantly higher occurrence among women than men. In rheumatoid arthritis, the membranes or tissues (synovial membranes) lining the joints become inflamed (synovitis). Over time, the inflammation may destroy the joint tissues, leading to disability. Because rheumatoid arthritis can affect multiple organs of the body, rheumatoid arthritis is referred to as a systemic illness and is sometimes called rheumatoid disease. The onset of rheumatoid disease is usually in middle age, but frequently occurs in one's 20s and 30s.

The pain and whole-body (systemic) symptoms associated with rheumatoid disease can be disabling. Over time, rheumatoid arthritis can cause significant joint destruction, leading to deformity and difficulty with daily activities. It is not uncommon for people with rheumatoid arthritis to suffer from some degree of depression, which may be caused by pain and progressive disability. A study reports that one-fourth of people with rheumatoid arthritis are unable to work by 6 to 7 years after their diagnosis, and half are not able to work after 20 years (O'Dell J R (2001). Rheumatoid arthritis: The clinical picture. In W J Koopman, ed., Arthritis and Allied Conditions: A Textbook of Rheumatology, 14th ed., vol. 1, chap. 58, pp. 1153-1186. Philadelphia: Lippincott Williams and Wilkins). Musculoskeletal conditions such as rheumatoid arthritis cost the U.S. economy nearly $65 billion per year in medical care and indirect expenses such as lost wages and production.

Synovial inflammation, rapid degradation of cartilage, and erosion of bone in affected joints are characteristic of rheumatoid arthritis (RA). Recent evidence indicates that skeletal tissue degradation and inflammation are regulated through overlapping but not identical biological processes in the rheumatoid joint and that therapeutic effects on these two aspects need not be correlated. Due to the complexity of the biological processes in the joint, mathematical and computer models can be used to help better understand the interactions between the various tissue compartments, cell types, mediators, and other factors involved in joint disease and healthy homeostasis. Several researchers have constructed simple models of the mechanical environment of the joint, rather than the biological processes of rheumatoid arthritis, and compared the results to patterns of disease and development in cartilage and bone (Wynarsky & Greenwald, J. Biomech., 16:241-251, 1983; Pollatschek & Nahir, J. Theor. Biol., 143:497-505, 1990; Beaupre et al., J. Rehabil. Res. Dev., 37:145-151, 2000; Shi et al., Acta Med. Okayama, 17:646-653, 1999). A computer manipulable mathematical model of joint diseases that includes multiple compartments including the synovial membrane and the interactions of these compartments is described in published PCT application WO 02/097706, published 5 Dec. 2002 and U.S. patent application Ser. No. 10/154,123, published 24 Apr. 2003 as 2003-0078759. Both publications are incorporated by reference in their entirety.

Rheumatoid arthritis is a chronic disease that, at present, can be controlled but not cured. The goal of treatment is relief of symptoms and keeping the disease from getting worse. The goals of most treatments for rheumatoid arthritis are to relieve pain, reduce inflammation, slow or stop the progression of joint damage, and improve a person's ability to function. Current approaches to treatment include lifestyle changes, medication, surgery, and routine monitoring and care. Medications used for the treatment of rheumatoid arthritis can be divided into two groups based on how they affect the progression of the disease: (1) symptom-relieving drugs and (2) disease-modifying drugs.

Medications to relieve symptoms, such as pain, stiffness, and swelling, may be used. Nonsteroidal anti-inflammatory drugs (NSAIDs), such as aspirin, ibuprofen, and naproxen are used to control pain and may help reduce inflammation. They do not control the disease or stop the disease from getting worse. Corticosteroids, such as prednisone and methylprednisolone (Medrol), are used to control pain and reduce inflammation. They may control the disease or stop the disease from getting worse; however, using corticosteroids as the only therapy for an extended time is not considered the best treatment. Corticosteroids are often used to control symptoms and flares of joint inflammation until anti-rheumatic drugs reach their full effectiveness, which can take up to 6 months. Nonprescription medications such as acetaminophen and topical medications such as capsaicin are used to control pain, but do not usually affect joint swelling or worsening of the disease.

Disease-modifying anti-rheumatic drugs (DMARDs) are used to control the progression of rheumatoid arthritis and to try to prevent joint deterioration and disability. These anti-rheumatic drugs are often given in combination with other anti-rheumatic drugs or with other medications, such as nonsteroidal anti-inflammatory drugs. Disease-modifying anti-rheumatic drugs commonly prescribed for rheumatoid arthritis include antimalarial medications such as hydroxycholoroquine (Plaquenil) or chloroquine (Aralen), methotrexate such as Rheumatrex, sulfasalazine (Azulfidine), leflunomide (Arava), etanercept (Enbrel), infliximab (Remicade), and anakinra (Kineret). DMARDs less commonly prescribed for rheumatoid arthritis include azathioprine (Imuran), penicillamine (e.g., Cuprimine or Depen), gold salts (e.g., Ridaura or Aurolate), minocycline (e.g., Dynacin or Minocin), cyclosporine (e.g., Neoral or Sandimmune), and cyclophosphamide (e.g., Cytoxan or Neosar). Some of these anti-rheumatic drugs can take up to 6 months to work. Many have serious side effects.

Thus a need exists for new, therapeutically effective drugs for the treatment of rheumatoid arthritis as well as new methods for identifying such drugs.

### D. SUMMARY OF THE INVENTION

In one aspect, the invention provides methods for alleviating at least one symptom of rheumatoid arthritis comprising administering a therapeutically effective amount of an antagonist of CD99 activity to a patient having rheumatoid arthritis. The antagonist of CD99 activity may be a protein, nucleic acid or small molecule inhibitor. A “small molecule” is defined herein as a molecule having a molecular weight of less than 1000 daltons. A preferred protein antagonist is a monoclonal antibody. Preferred nucleic acid antagonists include antisense inhibitors of mic2, the gene encoding CD99. In preferred embodiments, the patient is a methotrexate resistant patient, a TNF-α blockade cartilage nonresponder (CNR), a TNF-α blockade hyperplasia nonresponder (HNR), or a TNF-α blockade double nonresponder (DNR).

Another aspect of the invention provides methods of decreasing density of synovial cells in a joint comprising administering a therapeutically effective amount of an antagonist of CD99 activity to a patient having a condition associated with abnormally increased synovial cell density.

The invention also provides methods of decreasing cartilage degradation in a joint comprising administering a therapeutically effective amount of an antagonist of CD99 activity to a patient having a condition associated with an abnormally high rate of cartilage degradation.

An aspect of the invention provides methods of decreasing IL-6 concentration in synovial tissue comprising administering a therapeutically effective amount of an antagonist of CD99 activity to a patient having a condition associated with abnormally high concentration of IL-6 in synovial tissue.

Another aspect of the invention provides methods of decreasing bone erosion in a joint comprising administering a therapeutically effective amount of an antagonist of CD99 activity to a patient having a condition associated with an abnormally high rate of bone erosion.

In another aspect, the invention provides methods of alleviating at least one symptom of an inflammatory disease comprising administering a therapeutically effective amount of an antagonist of CD99 activity to a patient having an inflammatory disease. In preferred embodiments, the inflammatory disease is selected from the group consisting of diabetes, artheriosclerosis, inflammatory aortic aneurysm, restenosis, ischemia/reperfusion injury, glomerulonephritis, restenosis, reperfusion injury, rheumatic fever, systemic lupus erythematosus, rheumatoid arthritis, Reiter's syndrome, psoriatic arthritis, ankylosing spondylitis, coxarthritis, inflammatory bowel disease, ulcerative colitis, Crohn's disease, pelvic inflammatory disease, multiple sclerosis, diabetes, osteomyelitis, adhesive capsulitis, oligoarthritis, osteoarthritis, periarthritis, polyarthritis, psoriasis, Still's disease, synovitis, Alzheimer's disease, Parkinson's disease, amyotrophic lateral sclerosis, osteoporosis, and inflammatory dermatosis. More preferably the inflammatory disease is an arthritis, such as rheumatoid arthritis, psoratic arthritis, coxarthritis, osteoarthritis, or polyarthritis. Most preferably, the inflammatory disease is rheumatoid arthritis.

Yet another aspect of the invention provides methods of identifying a compound as an antagonist of CD99 activity comprising: (a) comparing an amount of leukocytes that migrate through at least one layer of endothelial cells in the presence of the compound with an amount of leukocytes that migrate through at least one layer of endothelial cells in the absence of the compound; and (b) identifying the compound as an antagonist of CD99 activity when the amount of migrating leukocytes in the presence of the compound is less than the amount of migrating leukocytes in the absence of the compound. In a preferred embodiment, the endothelial cells are cultured human umbilical vein endothelial cells, optionally stimulated with tumor necrosis factor of interleukins-1. In another preferred embodiment, the at least one layer of endothelial cells is a monolayers of endothelial cells. Another aspect of the invention includes methods of manufacturing a drug for use in the treatment of rheumatoid arthritis comprising identifying a antagonist of CD99 activity and formulating the identified antagonist for human consumption. In preferred embodiments, the leukocyte is either a monocyte or a T-cell.

The invention also provides methods of identifying a compound useful for the treatment of rheumatoid arthritis comprising: (a) comparing an amount of a T-cell cytokine produced by a first population of T-cells stimulated with a non-blocking anti-CD99 antibody in the presence of the compound with an amount of T-cell cytokine produced by a second population of T-cells in the absence of the compound; and (b) identifying the compound as useful for treatment of rheumatoid arthritis when the amount of T-cell cytokine produced in the presence of the compound is less than the amount of T-cell cytokine produced in the absence of the compound. In one embodiment of the invention, the T-cell cytokine is a Th1 cytokine. Preferably the Th1 cytokine that is measured is TNF-α or IFNγ. Another embodiment of the invention includes methods of manufacturing a drug for use in the treatment of rheumatoid arthritis comprising

identifying an antagonist of CD99 activity and formulating the identified antagonist for human consumption.

Another aspect of the invention provides methods of identifying a drug useful for the treatment of rheumatoid arthritis comprising: (a) comparing a number of a T-cells developed from a first population of T-cells stimulated with a non-blocking anti-CD99 antibody in the presence of the compound with a number of T-cells developed from a second population of T-cells in the absence of the compound, wherein the number of T-cells in the first population and second population are the same prior to stimulation with the non-blocking anti-CD99 antibody and exposure to the compound; and (b) identifying the compound as an antagonist of CD99 activity when the number of T-cells developed in the presence of the compound is less than the number of T-cells developed in the absence of the compound. Another embodiment of the invention includes methods of manufacturing a drug for use in the treatment of rheumatoid arthritis comprising identifying an antagonist of CD99 activity and formulating the identified antagonist for human consumption.

Yet another aspect of the invention provides methods of screening a collection of compounds for use in the treatment of rheumatoid arthritis comprising (a) comparing an amount of leukocytes that migrate through at least one layer of endothelial cells in the presence of a compound of the collection with an amount of leukocytes that migrate through at least one layer of endothelial cells in the absence of the compound; and (b) selecting the compound as an antagonist of CD99 activity when the amount of migrating leukocytes in the presence of the compound is less than the amount of migrating leukocytes in the absence of the compound. Preferably, these steps are repeated for each compound of the collection and at least one compound of the collection is selected as an antagonist of CD99 activity.

Yet another aspect of the invention provides methods of screening a collection of compounds for use in the treatment of rheumatoid arthritis comprising (a) comparing an amount of a Th1 cytokine produced by a first population of T-cells stimulated with a non-blocking anti-CD99 antibody in the presence of the compound with an amount of Th1 cytokine produced by a second population of T-cells in the absence of the compound; and (b) identifying the compound as an antagonist of CD99 activity when the amount of Th1 cytokine produced in the presence of the compound is less than the amount of Th1 cytokine produced in the absence of the compound. Preferably the Th1 cytokine that is measured is TNF-α or IFNγ.

An aspect of the invention provides methods of identifying a compound useful for treatment of an inflammatory disease comprising (a) comparing an amount of CD99 activity in the presence of the compound with an amount of CD99 activity in the absence of the compound; and (b) identifying the compound as useful for treatment of an inflammatory disease when the amount of CD99 activity in the presence of the compound is lower than the amount of CD99 activity in the absence of the compound.

### II. BRIEF DESCRIPTION OF THE FIGURES

For a better understanding of the nature and objects of some embodiments of the invention, reference should be made to the following detailed description taken in conjunction with the accompanying drawings, in which:

FIG. 1 demonstrates the sensitivity of synovial cell density to perturbation of a number of biological processes, including, inter alia, T-cell receptor stimulation, T-cell apoptosis rate, monocyte recruitment rate, interferon-gamma production, and monocyte/macrophage activation index.

FIG. 2 demonstrates the sensitivity of the rate of cartilage degradation to perturbation of a number of biological processes, including, including, inter alia, T-cell receptor stimulation, T-cell apoptosis rate, monocyte recruitment rate, interferon-gamma production, and monocyte/macrophage activation index.

FIG. 3 demonstrates the effect of CD99 blockade on synovial cell density.

FIG. 4 demonstrates the effect of CD99 blockade on cartilage degradation.

FIG. 5 demonstrates the effect of CD99 blockade on IL-6 in synovial tissue.

FIG. 6 demonstrates simulation of CD99 blockade on individual significant biological processes.

FIG. 7 illustrates the relative effect of CD99 blockade in a methotrexate resistant patient on monocyte extravasation, T-cell recruitment, T-cell proliferation and T-cell production of IFNγ.

FIG. 8 demonstrates the effect of CD99 blockade on synovial cell density in a methotrexate resistant patient.

FIG. 9 demonstrates the effect of CD99 blockade on cartilage degradation in a methotrexate resistant patient.

FIG. 10A illustrates the most likely relative effect of CD99 blockade in a TNF-α cartilage nonresponder on monocyte extravasation, T-cell recruitment, T-cell proliferation and T-cell production of IFNγ examining each process individually. FIG. 10B illustrates the relative effect of CD99 blockade in a TNF-α cartilage nonresponder on monocyte extravasation, T-cell recruitment, T-cell proliferation and T-cell production of IFNγ, examined by turning off one process at a time.

FIG. 11 demonstrates the effect of CD99 blockade on synovial cell density in a TNF-α cartilage nonresponder.

FIG. 12 demonstrates the effect of CD99 blockade on cartilage degradation in a TNF-α cartilage nonresponder.

FIG. 13A illustrates the most likely relative effect of CD99 blockade in a TNF-α hyperplasia nonresponder on monocyte extravasation, T-cell recruitment, T-cell proliferation and T-cell production of IFNγ examining each process individually. FIG. 13B illustrates the relative effect of CD99 blockade in a TNF-α hyperplasia nonresponder on monocyte extravasation, T-cell recruitment, T-cell proliferation and T-cell production of IFNγ, examined by turning off one process at a time.

FIG. 14 demonstrates the effect of CD99 blockade on synovial cell density in a TNF-α hyperplasia nonresponder.

FIG. 15 demonstrates the effect of CD99 blockade on cartilage degradation in a TNF-α a hyperplasia nonresponder.

FIG. 16A illustrates the most likely relative effect of CD99 blockade in a TNF-α double nonresponder on monocyte extravasation, T-cell recruitment, T-cell proliferation and T-cell production of IFNγ examining each process individually. FIG. 16B illustrates the relative effect of CD99 blockade in a TNF-α double nonresponder on monocyte extravasation, T-cell recruitment, T-cell proliferation and T-cell production of IFNγ, examined by turning off one process at a time.

FIG. 17 demonstrates the effect of CD99 blockade on synovial cell density in a TNF-α double nonresponder.

FIG. 18 demonstrates the effect of CD99 blockade on cartilage degradation in a TNF-α double nonresponder.

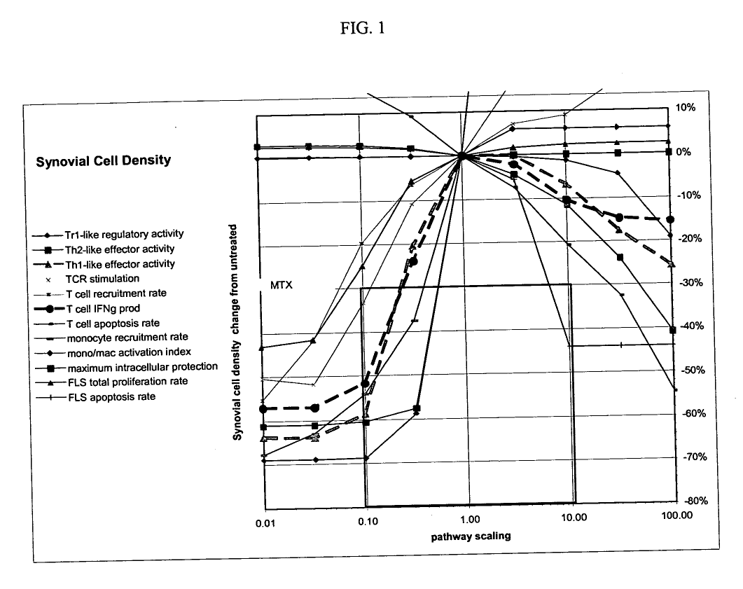

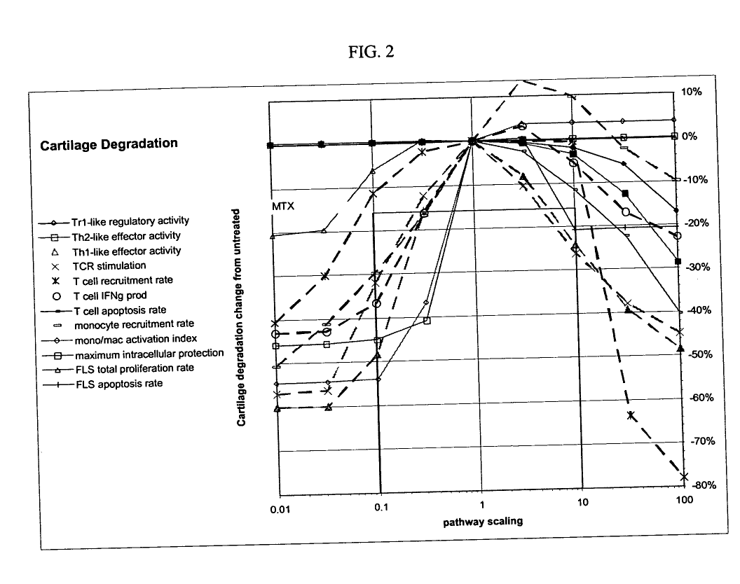

### III. DETAILED DESCRIPTION

A. Overview

In general this invention can be viewed as encompassing a novel method of treating inflammatory diseases, such as rheumatoid arthritis, and novel methods of identifying and screening for drugs useful in the treatment of inflammatory diseases and their clinical symptoms. Through the use of a computer model of a human rheumatic joint, the inventors have made the discovery that the activity of CD99, an adhesion molecule known to have an effect on some cancers, has a significant impact on the pathophysiology of rheumatoid arthritis. Inhibition of the activity of CD99 is predicted to alleviate the symptoms of inflammatory diseases, such as rheumatoid arthritis.

B. Definitions

The term “abnormally high concentration of IL-6 in synovial tissue,” as used herein, refers to a level of IL-6 in the synovial tissue of the diseased joint that is at least 3 standard deviations higher than that found in a normal, non-diseased, joint.

The term “abnormally high rate of bone erosion,” as used herein, refers to a detectable decrease in at least one dimension of a bone as determined by standard radiographic measures.

The term “abnormally high rate of cartilage degradation,” as used herein, refers to a detectable joint space narrowing as determined by standard radiographic measures. In a non-diseased joint narrowing is not detectable.

The term “abnormally increased synovial cell density,” as used herein, refers to a condition in which the synovial tissue of a joint contains a number of synovial cells that is at least ten-times higher than the number of synovial cells found in the synovial tissue of a normal, i.e., non-diseased, joint.

“Administering” means any of the standard methods of administering a pharmaceutical composition known to those skilled in the art. Examples include, but are not limited to intravenous, intramuscular or intraperitoneal administration.

The term “antagonist of CD99 activity,” as used herein, refers to the property of inhibiting any one of the four biological activities of CD99 shown to be relevant to rheumatoid arthritis: (1) monocyte recruitment, (2) T-cell proliferation, (3) T-cell activation and (4) T-cell recruitment. Inhibition need not be 100% effective in order to be antagonistic.

The term “drug” refers to a compound of any degree of complexity that can affect a biological system, whether by known or unknown biological mechanisms, and whether or not used therapeutically. Examples of drugs include typical small molecules of research or therapeutic interest; naturally-occurring factors such as endocrine, paracrine, or autocrine factors, antibodies, or factors interacting with cell receptors of any type; intracellular factors such as elements of intracellular signaling pathways; factors isolated from other natural sources; pesticides; herbicides; and insecticides. Drugs can also include, agents used in gene therapy such as DNA and RNA. Also, antibodies, viruses, bacteria, and bioactive agents produced by bacteria and viruses (e.g., toxins) can be considered as drugs. A response to a drug can be a consequence of, for example, drug-mediated changes in the rate of transcription or degradation of one or more species of RNA, drug-mediated changes in the rate or extent of translational or post-translational processing of one or more polypeptides, drug-mediated changes in the rate or extent of degradation of one or more proteins, drug-mediated inhibition or stimulation of action or activity of one or more proteins, and so forth. In some instances, drugs can exert their effects by interacting with a protein. For certain applications, drugs can also include, for example, compositions including more than one drug or compositions including one or more drugs and one or more excipients.

“Inflammatory diseases” refers to a class of diverse diseases and disorders that are characterized by any one of the following: the triggering of an inflammatory response; an upregulation of any member of the inflammatory cascade; the downregulation of any member of the inflammatory cascade. Inflammatory diseases include diabetes, artheriosclerosis, inflammatory aortic aneurysm, restenosis, ischemia/reperfusion injury, glomerulonephritis, restenosis, reperfusion injury, rheumatic fever, systemic lupus erythematosus, rheumatoid arthritis, Reiter's syndrome, psoriatic arthritis, ankylosing spondylitis, coxarthritis, inflammatory bowel disease, ulcerative colitis, Crohn's disease, pelvic inflammatory disease, multiple sclerosis, diabetes, osteomyelitis, adhesive capsulitis, oligoarthritis, osteoarthritis, periarthritis, polyarthritis, psoriasis, Still's disease, synovitis, Alzheimer's disease, Parkinson's disease, amyotrophic lateral sclerosis, osteoporosis, and inflammatory dennatosis. The singular term “inflammatory disease” includes any one or more diseases selected from the class of inflammatory diseases, and includes any compound or complex disease state wherein a component of the disease state includes a disease selected from the class of inflammatory diseases.

The term “joint,” as used herein, comprises the synovial tissue, synovial fluid, articular cartilage, bone tissues, and their cellular and extracellular composition, and the soluble mediators they contain.

The term “methotrexate nonresponder” refers to a rheumatoid arthritis patient who does not effectively respond to methotrexate treatment or who initially responds to methotrexate becomes refractory over time.

The term “TNF-α blockade cartilage nonresponder” refers to a rheumatoid arthritis patient with low initial TNF-α activity who shows decreased synovial hyperplasia, but minimal reduction in cartilage degradation in response to TNF-α blockade.

The term “TNF-α blockade hyperplasia nonresponder” refers to a rheumatoid arthritis patient with abnormally high or resistant levels of TNF-α activity who yields improvement in cartilage degradation but little decrease in synovial hyperplasia in response to TNF-α blockade.

The term “TNF-α blockade double nonresponder” refers to a rheumatoid arthritis patient with negligible initial TNF-α activity who shows poor response in both synovial hyperplasia and cartilage degradation in response to TNF-α blockade.

The term “patient” refers to any warm-blooded animal, preferably a human. Patients having rheumatoid arthritis can include, for example, patients that have been diagnosed with rheumatoid arthritis, patients that exhibit one or more of the symptoms associated with rheumatoid arthritis, or patients that are progressing towards or are at risk of developing rheumatoid arthritis.

As used herein, a “therapeutically effective amount” of a drug of the present invention is intended to mean that amount of the compound which will inhibit an increase in synovial cells in a rheumatic joint or decrease the rate of cartilage degradation in a rheumatic joint or decrease IL-6 concentration in synovial tissue or decrease the rate of bone erosion, and thereby cause the regression and palliation of the pain and inflammation associated with rheumatoid arthritis.

C. In Silico Modeling of a Rheumatoid Arthritis Joint

The present invention draws upon results obtained from an in silico model of an arthritic joint. The model provides a mathematical representation of the dynamic processes related to the biological state of a human joint afflicted with rheumatoid arthritis. The main compartments contained in the computer model represent synovial tissue and cartilage at the cartilage-pannus junction of this prototypical rheumatoid arthritis joint. The current model takes into account various biological variables related to the processes involved in cartilage metabolism, tissue inflammation, and tissue hyperplasia, including the following:
macrophage population dynamics including recruitment, activation, proliferation, apoptosis and their regulation,
T cell population dynamics including recruitment, antigen-dependent and antigen-independent activation, proliferation, apoptosis and their regulation
Fibroblast-like synoviocyte (FLS) population dynamic including influx in the tissue, proliferation, and apoptosis and their regulation
chondrocyte population dynamics including: proliferation and apoptosis
synthesis and regulation of a variety of proteins, including growth factors, cytokines, chemokines, proteolytic enzymes and matrix proteins, by the different cell type represented (macrophages, FLS, T cells, chondrocytes).
expression of adhesion molecules by endothelial cells
diffusion of mediator between synovial tissue and cartilage
interaction between cytokines or proteases and their natural inhibitors, antigen presentation, and
binding of therapeutic agents to cellular mediators (anti-TNF-α agents, such as etanercept and infliximab, and IL-1 RA antagonists, such as anakinra).
   The model also monitors synovial tissue density and the vascular volume. In addition, the mathematical model can take into account the effect of therapeutic agents such as methotrexate, steroids, non-steroidal anti-inflammatory drugs, soluble TNF-α receptor, TNF-α antibody, and interleukin-1 receptor antagonists.

In silico modeling is an approach that integrates relevant biological data—genomic, proteomic, and physiological—into a computer-based platform to reproduce a system's control principles. Given a set of initial conditions representing a defined disease state, these computer-based models can simulate the system's future biological behavior, a process termed biosimulation.

### 1. Top-Down Approach to Modeling Rheumatoid Arthritis

The computer model of the present invention was built using a “top-down” approach that started by defining a general set of behaviors indicative of rheumatoid arthritis. These behaviors are then used as constraints on the system and a set of nested subsystems is developed to define the next level of underlying detail. For example, given a behavior such as cartilage degradation in rheumatoid arthritis, the specific mechanisms inducing that behavior are each modeled in turn, yielding a set of subsystems, which themselves are deconstructed and modeled in detail. The control and context of these subsystems is, therefore, already defined by the behaviors that characterize the dynamics of the system as a whole. The deconstruction process continues modeling more and more biology, from the top down, until there is enough detail to replicate the known biological behavior of rheumatoid arthritis.

When using a top-down approach, public and proprietary data is identified and collected to support two specific purposes: (1) describing basic biology and (2) describing physiological function or behavior of the whole system. Data describing physiological functions or behavior of the whole system are selected early in the development of the model. These data represent the broad range of behaviors of the models system, i.e. cartilage degradation as a measurement (behavior) of rheumatoid arthritis patients. These data are human in vivo data based on well established clinical trials. Data describing basic biology is selected to sufficiently model the subsystems required to simulate the selected behaviors. These data can be human or animal (where human is preferred but not always available) in vivo, in vitro, or ex vivo data which provide an understanding of the underlying biology.

This modeling approach allows researchers to pose and rapidly develop “What if . . . ” scenarios by manipulating the biology at the subsystem level and observing simulated behaviors at the systems level. Through this approach, researchers can discover inconsistencies with commonly accepted, but yet unproven, hypotheses and identify key knowledge gaps from the tremendous amount of in vitro and in vivo data available to the scientist. When inconsistencies and knowledge gaps are identified, the model can be used to direct specific data collection efforts that are better focused, better designed, and the data they yield more predictive and efficiently utilized.

The top-down approach was used to develop a model of rheumatoid arthritis in a human joint. A similar model is described in co-pending U.S. patent application Ser. No. 10/154,123, published 24 Apr. 2003 as 2003-0078759. Four key clinical outcomes are of particular interest in the present model: synovial cell density, the rate of cartilage degradation, the level of IL-6 in synovial tissue and the rate of bone erosion. Rheumatoid arthritis is a systemic inflammatory disease with elevated levels of proinflammatory cytokines in peripheral blood, especially IL-6. C-reactive protein (CRP) is a common marker of inflammation which is routinely measured in the plasma, and several studies have shown a correlation between the concentration of IL-6 and the concentration of CRP in rheumatoid arthritis patients. Therefore, IL-6 concentration in either the joint or the plasma represents a good marker of inflammation.

2. Sensitivity Analysis

The explicit representation of the underlying biology of the disease allows the modulation of each subsystem alone or in combination to identify the one(s) with most impact on a specific clinical outcome, such as cartilage degradation or synovial cell density. By focusing modeling and data collection efforts on those subsystems with the greatest impact on the phenotypic onset and progression or rheumatoid arthritis, this approach can help more clearly represent the system's complexity and identify causal factors underlying the pathophysiology of rheumatoid arthritis. By modulating, in silico, each subsystem (e.g. knocking-out one cell type or intercellular mediator, or blocking one particular biological process), its contribution to the overall disease pathophysiology can be evaluated to better understand the biological phenomena driving rheumatoid arthritis, thus identifying the best and most relevant targets.

In the case of rheumatoid arthritis, the disease state can be represented as outputs associated with, for example, enzyme activities, product formation dynamics, and cellular functions that can indicate one or more biological processes that cause, affect, or are modified by the disease state. Typically, the outputs of the computer model include a set of values that represent levels or activities of biological constituents or any other behavior of the disease state. Based on these outputs, one or more biological processes can be designated as critical biological processes.

The computer model can be executed to represent a modification to one or more biological processes. In particular, a modification to a biological process can be represented in the computer model to identify the degree of connection (e.g., the degree of correlation) between the biological process and rheumatoid arthritis. For example, a modification to a biological process can be represented in the computer model to identify the degree to which the biological process causes, affects, or is modified by rheumatoid arthritis. A biological process can be identified as causing rheumatoid arthritis if a modification to this biological process is observed to produce symptoms associated with rheumatoid arthritis, i.e., increased synovial cell density, cartilage degradation and IL-6 levels in the synovial tissue. In some instances, a modification to a biological process can be represented in the computer model to identify the degree of connection between other biological processes and rheumatoid arthritis.

In some instances, identifying the set of biological processes can include sensitivity analysis. Sensitivity analysis can involve prioritization of biological processes that are associated with the disease state. Sensitivity analysis can be performed with different configurations of the computer model to determine the robustness of the prioritization. In some instances, sensitivity analysis can involve a rank ordering of biological processes based on their degree of connection to the disease state. Sensitivity analysis allows a user to determine the importance of a biological process in the context of the disease state. An example of a biological process of greater importance is a biological process that increases the severity of the disease state. Thus, inhibiting this biological process can decrease the severity of the disease state. The importance of a biological process can depend not only on the existence of a connection between that biological process and the disease state but also on the extent to which that biological process has to be modified to achieve a change in the severity of the disease state. In a rank ordering, a biological process that plays a more important role in the disease state typically gets a higher rank. The rank ordering can also be done in a reverse manner, such that a biological process that plays a more important role in the disease state gets a lower rank. Typically, the set of biological processes include biological processes that are identified as playing a more important role in the disease state.

During the process of sensitivity analysis of rheumatoid arthritis the activity of biological processes such as but not limited to monocytes recruitment, T-cell recruitment, cell apoptosis, and cytokine production are modulated (increased and decreased) in a computer model one a time. Biosimulation is then conducted and the consequence of the modulation of a single biological process at different level of stimulation or inhibition is assessed by measuring clinical outcomes such as, but not restricted to, cartilage degradation, synovial cell density and IL-6 levels. The outcome of this analysis identified the biological processes that have significant impact on the clinical outcomes.

In the present invention, sensitivity analysis identified four areas of the biology of rheumatoid arthritis having a significant impact on the disease pathophysiology: (1) macrophage apoptosis, (2) interferon-gamma production, (3) Th1 cell activation and (4) T-cell and monocyte recruitment.

3. Target Identification

Based on the effects of CD99 activity inhibition as predicted by the model described above, CD99 blockade is predicted to be an effective therapy for rheumatoid arthritis.

The effects of CD99 on monocyte recruitment, and T-cell proliferation, activation and recruitment were quantified and were explicitly represented in the computer model of rheumatoid arthritis. As the contribution of CD99 activity on each of these biological processes is not clearly characterized, a range of effects was defined in order to characterize the contribution of CD99 activity (Table 1). The “lower max effect” value represents the lowest effect documented taking in consideration possible redundancies with other proteins, the “upper max effect” is the maximal possible effect of CD99 activity on each biological process and the “most likely max effect” is the estimation of the realistic contribution of CD99 activity in each biological process, taking in consideration the in vivo environment and redundancies.

TABLE 1
Effect of CD99 Activity on Joint Model

| | Lower | Most likely | Upper |
|---|---|---|---|
| Hypothesis | max effect | max effect | max effect |
| monocyte recruitment | 66% | 88% | 88% |
| T cell proliferation | 0% | 0% | 40% |
| T cell activation | 0% | 0% | 84% |
| T cell recruitment | 20% | 40% | 88% |

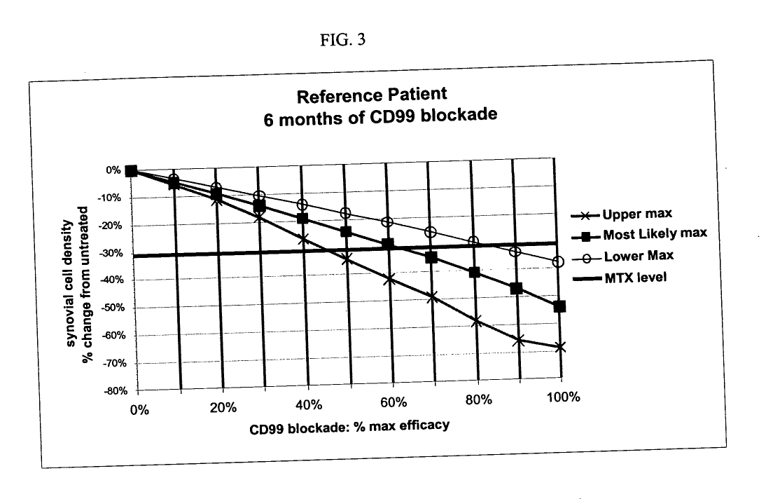

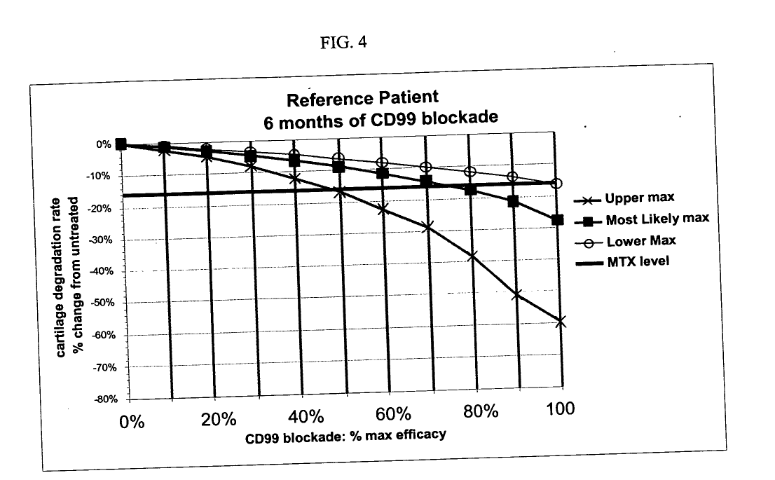

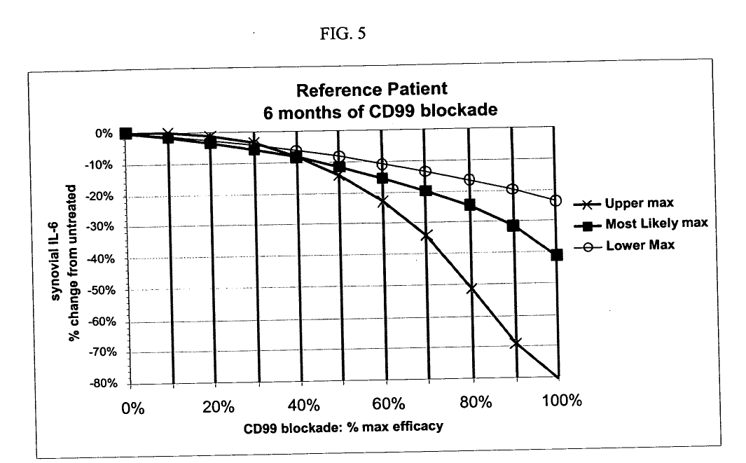

Simulation of the effect of CD99 activity on rheumatoid arthritis was then conducted by blocking CD99 in all relevant biological processes at once or in one biological process at time or in several biological processes in combination. The results of the simulation showed that blocking CD99 activity for 6 months could improve the rheumatoid arthritis clinical outcome by reducing cartilage degradation by 15 to 60%, synovial cell hyperplasia by 40 to 70% and IL-6 levels in synovial tissue by 16 to 60%. FIG. 3 demonstrates the effect of CD99 blockade on synovial cell density. FIG. 4 demonstrates the effect of CD99 blockade on cartilage degradation. FIG. 5 demonstrates the effect of CD99 blockade on IL-6 levels in synovial tissue.

Methotrexate is a common treatment for rheumatoid arthritis. Methotrexate treatment is known to decrease synovial cell density by approximately 30%, decrease the rate of cartilage degradation by approximately 15% and decrease the concentration of IL-6 in synovial tissue by 93%. At 100% efficacy, the computer model predicts CD99 antagonism will induce a greater improvement than methotrexate. The model predicts that compounds causing only a 70% of an inhibition of CD99 activity would be superior that methotrexate in decreasing synovial cell density and would have approximately the same or superior effect on the rate of cartilage degradation.

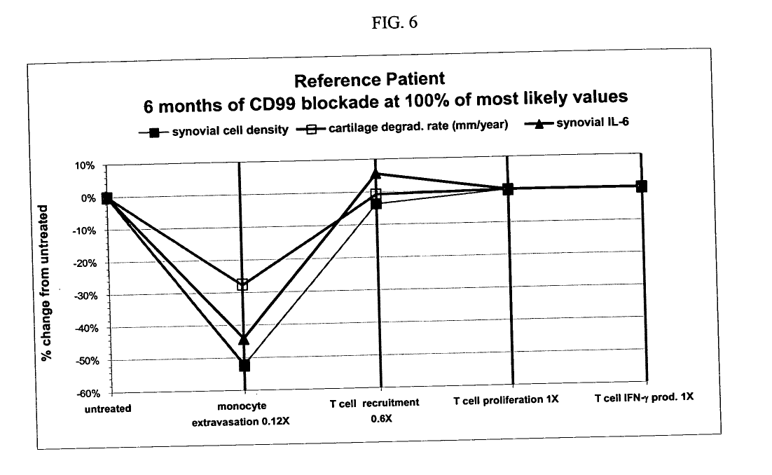

The simulation of CD99 blockade in one biological process at a time demonstrated that the main biological process driving the impact of CD99 blockade on the clinical outcome is the effect on monocyte recruitment. The computer model reveals that the effect of CD99 blockade on monocyte recruitment is responsible for more than 90% of the clinical improvement observed. FIG. 6 provides the relative effect of CD99 blockade on monocyte extravasation (monocyte recruitment), T-cell recruitment, T-cell proliferation and T-cell production of IFNγ.

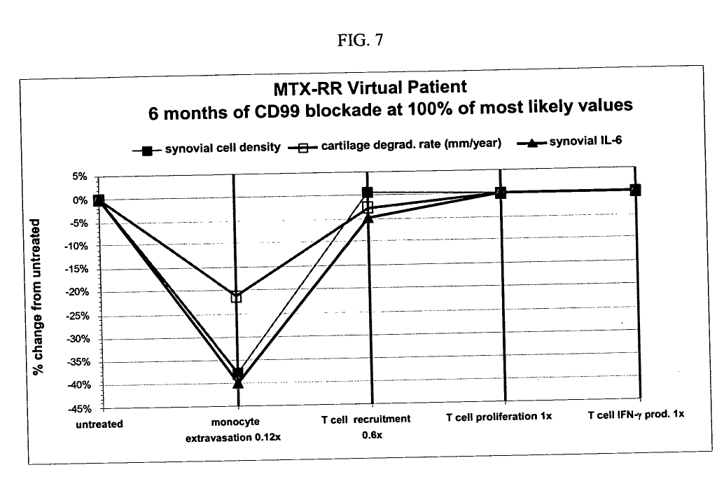

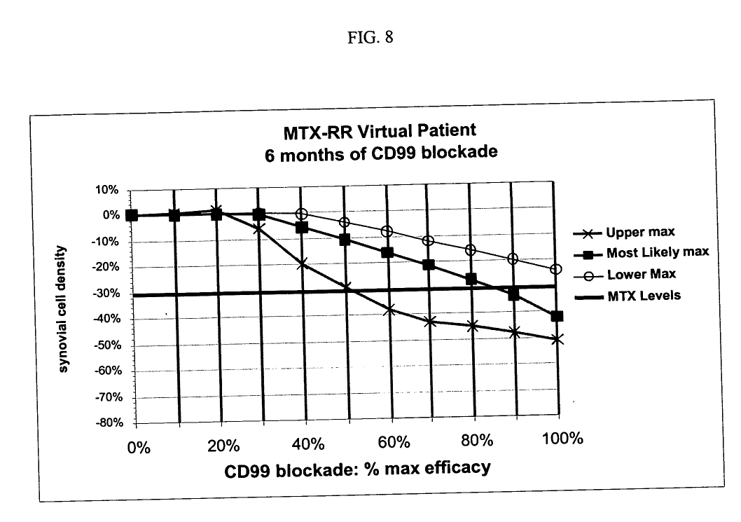

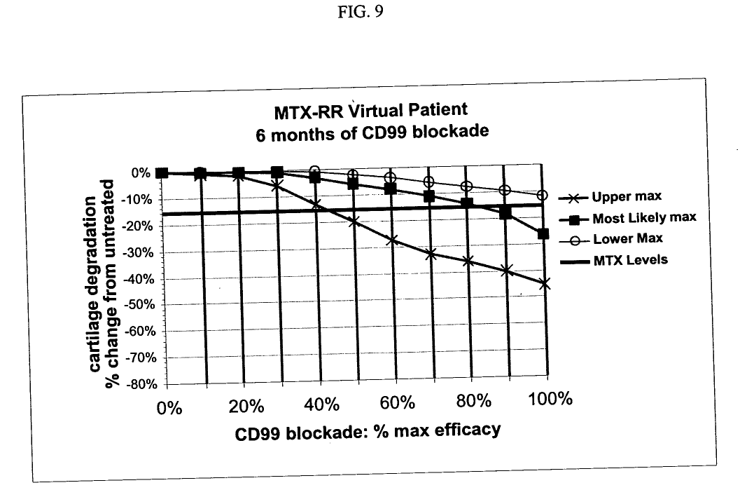

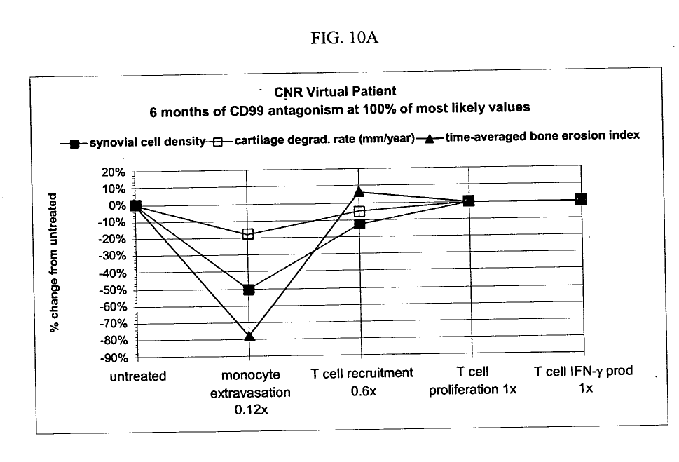

Some rheumatoid arthritis patients do not effectively respond to methotrexate treatment (initial nonresponders), while other patients who initially responded to methotrexate become refractory over time (gradual nonresponders). Simulation of blockading CD99 activity in a methotrexate resistant patient reveals a different pattern of response than in a non-resistant patient. FIG. 7 illustrates the relative effect of CD99 blockade in a methotrexate resistant patient on monocyte extravasation, T-cell recruitment, T-cell proliferation and T-cell production of IFNγ. In methotrexate resistant patients, CD99 activity in T-cell recruitment plays a substantially greater role in causing the symptoms of rheumatoid arthritis as compared to a non-resistant rheumatoid arthritis patient (cf FIG. 6). The results of the simulation showed that blocking CD99 activity for 6 months in a methotrexate resistant patient could improve the rheumatoid arthritis clinical outcome by reducing cartilage degradation by 12 to 45%, and synovial cell hyperplasia by 25 to 50%. FIG. 8 demonstrates the effect of CD99 blockade on synovial cell density in a methotrexate resistant patient. FIG. 9 demonstrates the effect of CD99 blockade on cartilage degradation in a methotrexate resistant patient.

TNF-α neutralizing therapies have become increasingly important in treating rheumatoid arthritis patients. However, roughly a third of all rheumatoid arthritis patients fail to achieve a clinically significant response to TNF-α neutralizing therapies. Three potential classes of TNF-α blockade nonresponders were defined in the model described above. Synovial hyperplasia and cartilage degradation are differentially affected when TNF-α varies within different ranges, leading to the identification of three nonresponder classes within the current model. Specifically, patients with low initial TNF-α activity show decreased synovial hyperplasia, but minimal reduction in cartilage degradation in response to TNF-α blockade (cartilage nonresponders, or CNRs), while patients with negligible initial TNF-α activity show poor response in both synovial hyperplasia and cartilage degradation (double nonresponders or DNRs). Alternatively, insufficient neutralization of TNF-α in patients with abnormally high or resistant levels of TNF-α activity yields improvement in cartilage degradation but poor response in hyperplasia (hyperplasia nonresponders or HNRS). Mechanistically, in patients with low levels of TNF-α, disease was perpetuated by increased activity of alternate macrophage activating pathways (e.g., CD40-ligation), reduced activity of anti-inflammatory cytokines (e.g., IL-10), and increased activity of degradation-promoting cytokines (e.g., IL-1b). Nonresponding patients also showed altered responses to other therapies such as IL-1Ra (data not shown).

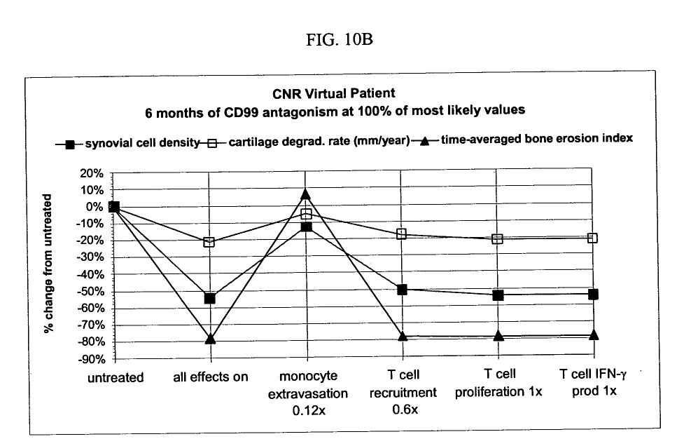

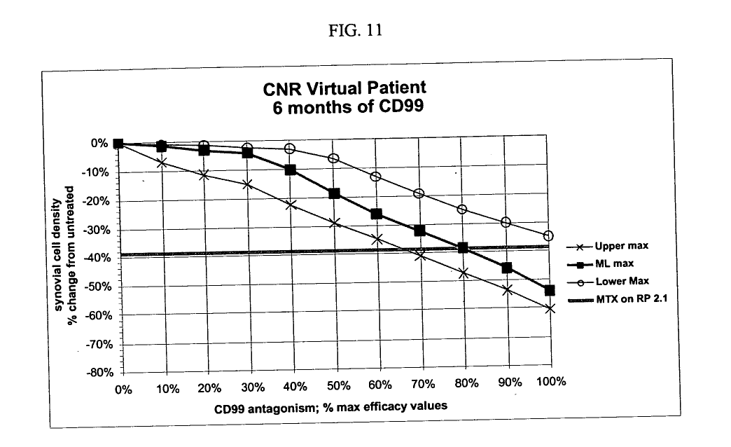

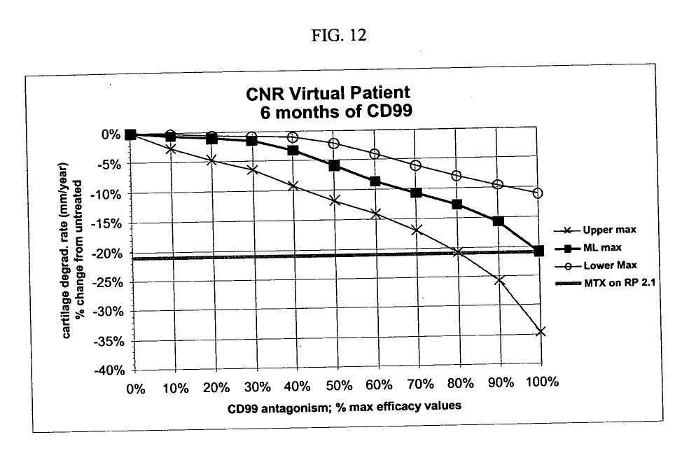

Simulation of blocking CD99 activity in a TNF-α-blockade CNR patient reveals a similar pattern of response to that in a non-resistant patient. FIGS. 10A and 10B illustrate the relative effect of CD99 blockade in a TNF-α-blockade CNR patient on monocyte extravasation, T-cell recruitment, T-cell proliferation and T-cell production of IFNγ. The results of the simulation showed that completely blocking CD99 activity for 6 months in a TNF-α-blockade CNR patient could improve the rheumatoid arthritis clinical outcome by reducing cartilage degradation by 11 to 35% and synovial cell hyperplasia by 35 to 61%. FIG. 11 demonstrates the effect of CD99 blockade on synovial cell density in a TNF-α-blockade CNR patient. FIG. 12 demonstrates the effect of CD99 blockade on cartilage degradation in a TNF-αα-blockade CNR patient.

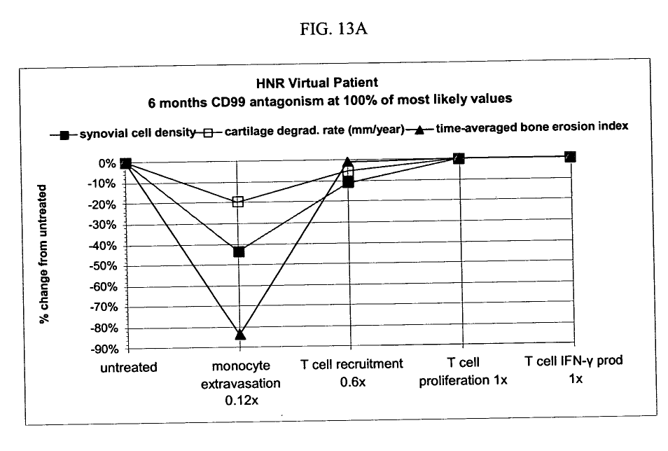

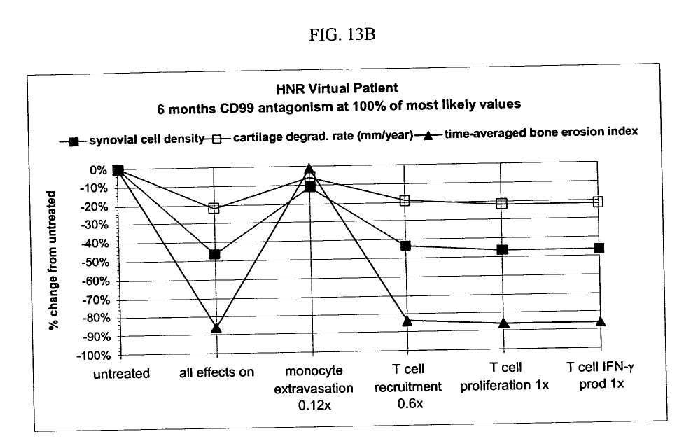

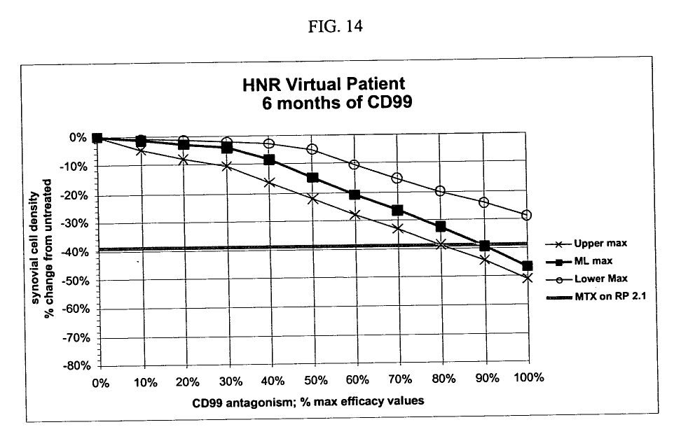

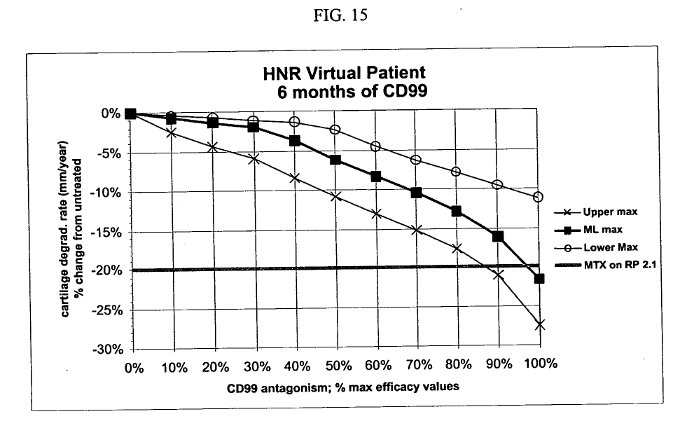

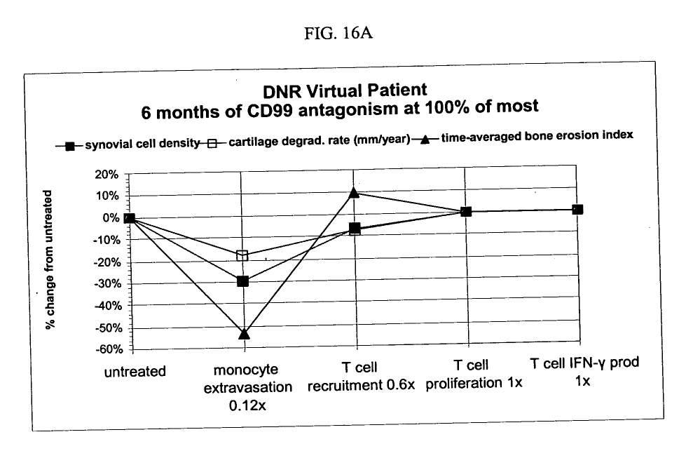

Simulation of blocking CD99 activity in a TNF-α-blockade HNR patient reveals a similar pattern of response to that in a non-resistant patient. FIGS. 13A and 13B illustrate the relative effect of CD99 blockade in a TNF-α-blockade HNR patient on monocyte extravasation, T-cell recruitment, T-cell proliferation and T-cell production of IFNγ. The results of the simulation showed that completely blocking CD99 activity for 6 months in a TNF-α-blockade HNR patient could improve the rheumatoid arthritis clinical outcome by reducing cartilage degradation by 11 to 27%, and synovial cell hyperplasia by 29 to 51%. FIG. 14 demonstrates the effect of CD99 blockade on synovial cell density in a TNF-α-blockade HNR patient. FIG. 15 demonstrates the effect of CD99 blockade on cartilage degradation in a TNF-α-blockade HNR patient.

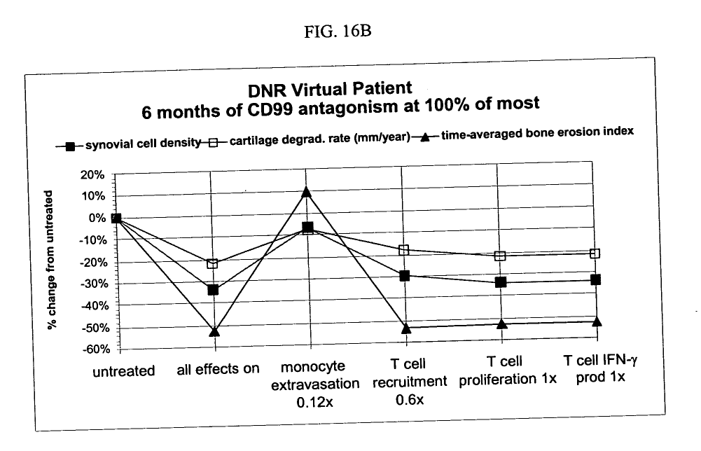

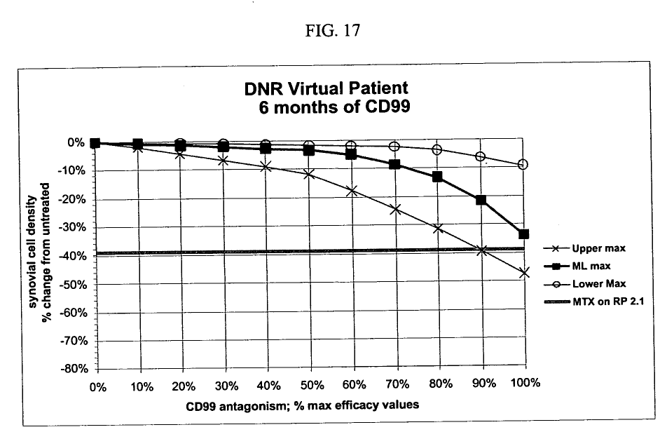

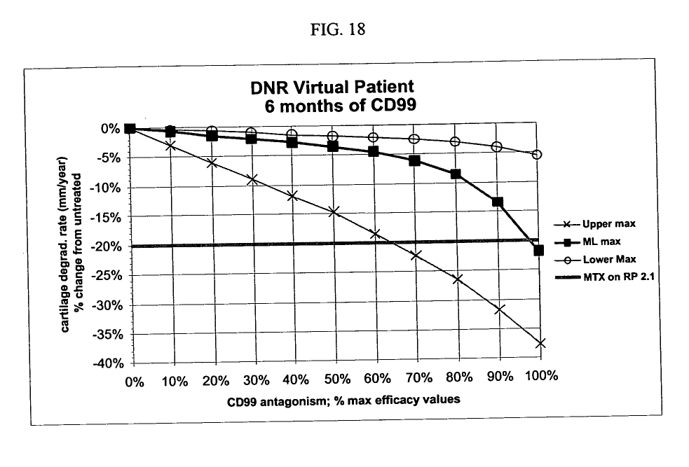

Simulation of blocking CD99 activity in a TNF-α-blockade DNR patient reveals a similar pattern of response to that in a non-resistant patient. FIGS. 16A and 16B illustrate the relative effect of CD99 blockade in a TNF-α-blockade DNR patient on monocyte extravasation, T-cell recruitment, T-cell proliferation and T-cell production of IFNγ. The results of the simulation showed that completely blocking CD99 activity for 6 months in a TNF-α-blockade DNR patient could improve the rheumatoid arthritis clinical outcome by reducing cartilage degradation by 6 to 38%, and synovial cell hyperplasia by 9 to 47%. FIG. 17 demonstrates the effect of CD99 blockade on synovial cell density in a TNF-α-blockade DNR patient. FIG. 18 demonstrates the effect of CD99 blockade on cartilage degradation in a TNF-α-blockade DNR patient.

Application of the in silico model of rheumatoid arthritis provided the surprising result that antagonism of CD99 activity is a promising therapeutic strategy for patients suffering from rheumatoid arthritis.

D. CD99

CD99 (also called MIC2 or E2 antigen) is a 32 kD cell-surface (transmembrane) glycoprotein encoded by the mic2 gene. The mic2 gene predicts a type I transmembrane protein of 16.7 kD that spans the cellular membrane once. There are no consensus N-linked glycosylation sites, but several sites for O-linked glycosylation, which accounts for 14 kD of CD99's apparent molecular weight of 32 kD. CD99 is not a member of any known protein family. CD99 is an adhesion molecule expressed mostly on monocytes, peripheral T cells (RO+ only), B cells, and thymocytes.

CD99 has been demonstrated to have an effect in various cancers such as Ewing's sarcoma (Scotlandi, 2000), Hodgkin's and Reed Sternberg B cells (Kim, Blood 2000) and breast cancer (Lee, 2002). CD99's function has best been characterized on T-cells, where it was found to be an alternative ligand to CD2 for the phenomenon of sheep blood cell rosetting. In addition, ligation of CD99 on thymocytes and T cells has been shown to play a co-stimulatory function in certain in vitro systems. Activity of CD99 has not been described in the literature as being relevant to rheumatoid arthritis.

Although its function is still under investigation, some important effects of CD99, mediated via homotypic binding, could be relevant in the context of rheumatoid arthritis. CD99 has been shown to play a critical role in diapedesis, or transendothelial migration, a step in the cascade of events leading to cell recruitment through the endothelial cells where CD99 acts as an adhesion molecule. Monocytes diapedesis is strongly dependent on CD99 activity. Generally, diapedesis occurs when endothelial cells are activated, e.g., with TNF-α, IL-1 or other pro-inflammatory mediators. Transendothelial migration also occurs endogenously usually at a lower level across endothelial cells as a result of leukocyte adhesion even in the absence of direct activation of the endothelial cells. Thus migration occurs in vivo at inflammatory foci. CD99 may also be involved in recruitment of monocytes (Schenkel, 2002).

CD99 also plays an indirect role in T-cell vascular adhesion via up-regulation of VCAM-1. CD99 has also been implicated in T-cell signaling (Waclavicek 1998, Wingott 1999, Bernard 2000), recruitment of T-cells (Bernard, 2000), cytokine production and activation by T-cells (Waclavicek 1998, Wingott 2000) and apoptosis of thymocytes (Petterson).

Numerous mouse and rat monoclonal antibodies have been developed against different epitopes of CD99 but only three have been tested for the blockade of CD99. These are Hec2 (Schenkel 2003), 0662 (Bernard 1995, 1997), and D44 (Bernard-Boumsell). Each of these antibodies inhibits transendothelial migration to some extent. Other anti-CD99 antibodies that can be used for the present invention include, but are not limited to: Hec2, D44, O662, MEM-131, TU12, HO36-1.1, HIT4, 013, N-16, C-20, B-N24, HI142, HI175, FMC29, HI147, HI170, L129, and Ad20. Antisense RNA inhibitors have also been demonstrated to antagonize the activity of CD99. (Kim, et al., Blood 92:4287-4295 (1998)). Epstein Barr virus latent protein 1 (LMP-1) has also been shown to directly cause down regulation of surface CD99 expression and therefore activity (Kim et al., Blood 95:294-300 (2000)).

E. Methods of Identifying CD99 Antagonists and Anti-Rheumatic Drugs

1. Monocyte Recruitment

One preferred assay for identifying antagonists of CD99 activity is a modification of a typical transmigration assay. Monocytes are in suspension above an endothelial layer growing on a porous support above a lower well of endogenous (made by the endothelium) or exogenous chemoattractant. The monocytes that end up in the lower chamber at the end of the assay are counted as transmigrated. Compounds that inhibit the activity of CD99 will decrease the number of cells that migrate across the endothelial layer.

In one preferred assay, endothelial cells are cultured on hydrated Type I collagen gels overlaid with fibronectin. Components of the culture medium penetrate into the porous gel. Alternatively, the endothelial cells may be grown on the upper surface of a porous filter suspended above a lower chamber. Culture medium is placed in the upper and lower chambers to reach the apical and basal surfaces of the monolayer. Monocytes are added to the upper chamber. In order to be counted as “migrated”, a monocyte must (1) attach to the apical surface of the endothelial cells, (2) migrate to the intercellular junction, (3) diapedese between the endothelial cells, (4) detach from the endothelial cells and penetrate the basal lamina, (5) cross the filter or gel and (6) detach from the filter or gel and enter the lower chamber.

Monocytes or neutrophils, freshly isolated from peripheral blood of healthy or rheumatic donor are allowed to settle on confluent endothelial monolayers at 37° C. in the presence or absence of test compounds. The assays may be run in a variety of media including, but not limited to complete medium, Medium 199, or RPMI1640, optionally supplemented with human serum albumin. After sufficient time for transendothelial migration, generally one hour, the monolayers are washed with a chelator, such as EGTA, to remove any monocytes or neutrophils still attached to the apical surface. If a collagen gel is used as a substrate, the monolayer is then rinsed with phosphate buffered saline with divalent cations and fixed in glutaraldehyde overnight. Fixing strengthens the collagen gel so that it is easier to manipulate. The monolayers are stained, preferably with Wright-Giemsa, and mounted on slides for direct observation, preferably under Nomarski optics. Using Nomarski optics, one can distinguish by the plane of focus, monocytes or neutrophils that are attached to the apical surface of the monolayer from those that have transmigrated. A quantifiable measure of transmigration is the percentage of those monocytes or neutrophils associated with the monolayer that have migrated beneath the monolayer. Therefore, the measurement of transmigration is independent of the degree of adhesion to the monolayer.

Migration of monocytes or neutrophils can be determined in the presence or absence of cytokine stimulation of the endothelium. Activation of endothelial cells can result from contact with stimulatory mediators. For the purpose of the present invention, activation of endothelial cells preferably results from contact with cytokines such as tumor necrosis factor (TNF) and interleukin-1 (IL-1).

The term “endothelial cell” has ordinary meaning in the art. Endothelial cells make up endothelium, which is found inter alia in the lumen of vascular tissue (veins, arteries, and capillaries) throughout the body. In arthritis leukocytes migrate from the circulating blood to the arthritic joint where they participate in inflammation.

2. T-Cell Recruitment

A similar transmigration assay can be applied to T lymphocytes freshly isolated from peripheral blood of healthy or rheumatic donor. After isolation, purified T lymphocytes are allowed to settle on confluent endothelial monolayers at 37° C. in the presence or absence of test compounds. The assays may be run in a variety of media including, but not limited to complete medium, Medium 199, or RPMI1640, optionally supplemented with human serum albumin. After sufficient time for transendothelial migration, generally one hour, the monolayers are washed with a chelator, such as EGTA, to remove any T-cells still attached to the apical surface. If a collagen gel is used as a substrate, the monolayer is then rinsed with phosphate buffered saline with divalent cations and fixed in glutaraldehyde overnight. Fixing strengthens the collagen gel so that it is easier to manipulate. The monolayers are stained, preferably with Wright-Giemsa and mounted on slides for direct observation, preferably under Nomarski optics. Using Nomarski optics, one can distinguish by the plane of focus, T-cells that are attached to the apical surface of the monolayer from those that have transmigrated. A quantifiable measure of transmigration is the percentage of those T-cells associated with the monolayer that have migrated beneath the monolayer.

Migration of T-cells can be determined in the presence or absence of cytokine stimulation of the endothelium. Activation of endothelial cells can result from contact with stimulatory mediators. For the purpose of the present invention, activation of endothelial cells preferably results from contact with cytokines such as tumor necrosis factor (TNF) and interleukin-1 (IL-1).

3. In Vitro T-Cell Proliferation

A preferred method for evaluating antagonists of CD99 activity is through the use of a T-cell activation assay, where the activation is defined by proliferation of T-cells. T cells are derived from healthy subjects or rheumatoid arthritis patients. Preferably, Th1 cells are obtained, however, any T-cell population may be used.

Proliferation of the T-cells is induced by stimulating the T-cells with an anti-CD99 antibody that activates CD99 on the T-cell without blocking normal binding and other physiological activities of CD99. 12E7 and 3B2/TA8 are known to activate CD99 without blocking it. Optionally, an anti-CD3 antibody such as, e.g., OKT3 may be added with the anti-CD99 antibody to potentiate stimulation of the T-cells. In addition, chemical stimulants such as PMA or ionomycin, optionally, may also be used. After the T-cells are stimulated, the cells are pulsed with a DNA label, such as tritiated thymidine [methyl-3H]TdR (Amershan). Alternatively, the number of cells can be directly counted. The cells are incubated for 12-24 hours, harvested and the radioactivity counted. The amount of radioactivity correlates to the number of viable T-cells.

The inhibition of T cell proliferation can be then assessed by incubating the T cells for 12-24 hours with both a stimulatory antibody (12E7 and 3B2/TA8 for example) and an antagonist of CD99 activity (antibody or other molecule). The residual proliferation of the purified T cells can be assessed using the assay previously described.

4. T-Cell Activation

Another method for evaluating antagonists of CD99 activity is through the use of a T-cell activation assay. T-cell activation can be assessed by measuring the production of cytokine after stimulation of the T-cell via a an anti-CD99 antibody that activates CD99 on the T-cell without blocking normal binding and other physiological activities of CD99. 12E7 and 3B2/TA8 are known to activate CD99 without blocking it. Optionally, an anti-CD3 antibody such as, e.g., OKT3 may be added with the anti-CD99 antibody to potentiate stimulation of the T-cells. In addition, chemical stimulants such as PMA or ionomycin, optionally, may also be used. Culture supernatants are then harvested and cytokine concentration measured using a method such as, but not limited to, sandwich ELISA.

The inhibition of T-cell activation (as measured by cytokine release) can be assessed by incubating the T-cells with both a stimulatory antibody (12E7 and 3B2/TA8 for example) and an antagonist of CD99 activity (antibody or other molecule). The residual cytokine production of the purified T cells can be assessed using the assay previously described.

The term “T cell” has ordinary meaning in the art. T cells are a class of lymphocytes, so called because they are derived from the thymus and have been through thymic processing. These cells are primarily involved in controlling cell-mediated immune reactions and in the control of B-cell development. The T-cells coordinate the immune system by secreting lymphokine hormones.

F. Methods of Treatment

In another aspect, the invention provides methods of alleviating at least one symptom of an inflammatory disease, such as rheumatoid arthritis, comprising administering a therapeutically effective amount of an antagonist of CD99 activity to a patient having an inflammatory disease. The invention also provides methods for alleviating at least one symptom of rheumatoid arthritis comprising administering a therapeutically effective amount of an antagonist of CD99 activity to a patient having rheumatoid arthritis. The antagonist of CD99 activity may be a protein, nucleic acid or small molecule inhibitor. A preferred protein antagonist is an antibody, more preferably a monoclonal antibody. Preferred nucleic acid antagonists include antisense inhibitors of mic2, the gene encoding CD99. The invention also encompasses methods of decreasing synovial cell density, methods of decreasing cartilage degradation and methods of decreasing IL-6 concentration in synovial tissue by administering a therapeutically effective amount of an antagonist of CD99 activity.

A compound useful in this invention is administered to a patient in a therapeutically effective dose by a medically acceptable route of administration such as orally, parentally (e.g., intramuscularly, intravenously, subcutaneously, interperitoneally), transdermally, rectally, by inhalation and the like. The dosage range adopted will depend on the route of administration and on the age, weight and condition of the patient being treated.

Various delivery systems are known and can be used to administer a composition of the invention, e.g., encapsulation in liposomes, microparticles, microcapsules, recombinant cells capable of expressing the compound, receptor-mediated endocytosis (see, e.g., Wu and Wu, 1987, J. Biol. Chem. 262:4429-4432), construction of a nucleic acid as part of a retroviral or other vector, etc. Methods of introduction include, but are not limited to, intradermal, intramuscular, intraperitoneal, intravenous, subcutaneous, intranasal, epidural, and oral routes. The compositions may be administered by any convenient route, for example by infusion or bolus injection, by absorption through epithelial or mucocutaneous linings (e.g., oral mucosa, rectal and intestinal mucosa, etc.) and may be administered together with other biologically active agents. Administration can be systemic or local. In addition, it may be desirable to introduce the compositions of the invention into the central nervous system by any suitable route, including intraventricular and intrathecal injection; intraventricular injection may be facilitated by an intraventricular catheter, for example, attached to a reservoir, such as an Ommaya reservoir. Pulmonary administration can also be employed, e.g., by use of an inhaler or nebulizer, and formulation with an aerosolizing agent.

G. Pharmaceutical Compositions

1. Antibodies

Antibodies of the invention include, but are not limited to, polyclonal, monoclonal, bispecific, human, humanized or chimeric antibodies, single chain antibodies, sFvs fragments, F(ab′) fragments, fragments produced by a Fab expression library, anti-idiotypic (anti-Id) antibodies, and epitope-binding fragments of any of the above which immunospecifically bind to CD99 and cells expressing CD99. The term “antibody” as used herein refers to immunoglobulin molecules and immunologically active portions of immunoglobulin molecules, i.e., molecules that contain an antigen binding site which immunospecifically binds CD99 and/or cells expressing CD99. The immunoglobulin molecules of the invention can be of any type (e.g., IgG, IgE, IgM, IgD and IgA), class, or subclass of immunoglobulin molecule.

Polyclonal antibodies which may be used in the methods of the invention are heterogeneous populations of antibody molecules derived from the sera of immunized animals. Various procedures, well known in the art, may be used for the production of polyclonal antibodies to CD99. For example, for the production of polyclonal antibodies, various host animals, including but not limited to rabbits, mice, rats, etc., can be immunized by injection with CD99 or a derivative thereof. Various adjuvants may be used to increase the immunological response, depending on the host species, and including but not limited to Freund's (complete and incomplete), mineral gels such as aluminum hydroxide, surface active substances such as lysolecithin, pluronic polyols, polyanions, peptides, oil emulsions, keyhole limpet hemocyanins, dinitrophenol, and potentially useful human adjuvants such as BCG (bacille Calmette-Guerin) and corynebacterium parvum. Such adjuvants are also well known in the art.

Monoclonal antibodies which may be used in the methods of the invention are homogeneous populations of antibodies to a particular antigen (e.g., CD99). For the purposes of this invention a “monoclonal antibody” is an antibody produced by a hybridoma cell. Methods of making monoclonal antibody-synthesizing hybridoma cells are well known to those skilled in the art, e.g., by the fusion of an antibody producing B lymphocyte with an immortalized B-lymphocyte cell line. Preferably the monoclonal antibody will be a murine monoclonal antibody, a chimeric monoclonal antibody, a humanized monoclonal antibody, or, most preferably, a human monoclonal antibody.

A monoclonal antibody (mAb) to CD99 can be prepared by using any technique known in the art which provides for the production of antibody molecules by continuous cell lines in culture. These include but are not limited to, the hybridoma technique originally described by Kohler and Milstein (1975, Nature 256, 495-497), the more recent human B cell hybridoma technique (Kozbor et al., 1983, Immunology Today 4:72), and the EBV-hybridoma technique (Cole et al., 1985, Monoclonal Antibodies and Cancer Therapy, Alan R. Liss, Inc., pp. 77-96). Such antibodies may be of any immunoglobulin class including IgG, IgM, IgE, IgA and, IgD and any subclass thereof. The hybridoma producing the mAbs of use in this invention may be cultivated in vitro or in vivo.

The monoclonal antibodies which may be used in the methods of the invention include, but are not limited to, human monoclonal antibodies or chimeric human-mouse (or other species) monoclonal antibodies. Human monoclonal antibodies may be made by any of numerous techniques known in the art (e.g., Teng et al., 1983, Proc. Natl. Acad. Sci. U.S.A. 80, 7308-7312; Kozbor et al., 1983, Immunology Today 4, 72-79; and Olsson et al., 1982, Meth. Enzymol. 92, 3-16).

The invention provides for the use of functionally active fragments, derivatives or analogs of antibodies which immunospecifically bind to CD99 and/or cells expressing CD99. Functionally active means that the fragment, derivative or analog is able to elicit anti-anti-idiotype antibodies that recognize the same antigen that the antibody from which the fragment, derivative or analog is derived recognized. Specifically, in a preferred embodiment the antigenicity of the idiotype of the immunoglobulin molecule may be enhanced by deletion of framework and CDR sequences that are C-terminal to the CDR sequence that specifically recognizes the antigen. To determine which CDR sequences bind the antigen, synthetic peptides containing the CDR sequences can be used in binding assays with the antigen by any binding assay method known in the art (e.g., the BIAcore assay)

Other embodiments of the invention include fragments of the antibodies of the invention such as, but not limited to, F(ab′)₂ fragments, which contain the variable region, the light chain constant region and the CH1 domain of the heavy chain can be produced by pepsin digestion of the antibody molecule, and Fab fragments, which can be generated by reducing the disulfide bridges of the F(ab′)₂ fragments. The invention also provides heavy chain and light chain dimers of the antibodies of the invention, or any minimal fragment thereof such as Fvs or single chain antibodies (SCAs) (e.g., as described in U.S. Pat. No. 4,946,778; Bird, 1988, Science 242:423-42; Huston et al., 1988, Proc. Natl. Acad. Sci. USA 85:5879-5883; and Ward et al., 1989, Nature 334:544-54), or any other molecule with the same specificity as the antibody of the invention.

Additionally, recombinant antibodies, such as chimeric and humanized monoclonal antibodies, comprising both human and non-human portions, which can be made using standard recombinant DNA techniques, are within the scope of the invention. A chimeric antibody is a molecule in which different portions are derived from different animal species, such as those having a variable region derived from a murine monoclonal and a human immunoglobulin constant region. (See, e.g., Cabilly et al., U.S. Pat. No. 4,816,567; and Boss et al., U.S. Pat. No. 4,816,397, which are incorporated herein by reference in their entirety.) Humanized antibodies are antibody molecules from non-human species having one or more complementarily determining regions (CDRs) from the non-human species and a framework region from a human immunoglobulin molecule. (See, e.g., Queen, U.S. Pat. No. 5,585,089, which is incorporated herein by reference in its entirety.) Such chimeric and humanized monoclonal antibodies can be produced by recombinant DNA techniques known in the art, for example using methods described in PCT Publication No. WO 87/02671; European Patent Application 184,187; European Patent Application 171,496; European Patent Application 173,494; PCT Publication No. WO 86/01533; U.S. Pat. No. 4,816,567; European Patent Application 125,023; Better et al., 1988, Science 240:1041-1043; Liu et al., 1987, Proc. Natl. Acad. Sci. USA 84:3439-3443; Liu et al., 1987, J. Immunol. 139:3521-3526; Sun et al., 1987, Proc. Natl. Acad. Sci. USA 84:214-218; Nishimura et al., 1987, Canc. Res. 47:999-1005; Wood et al., 1985, Nature 314:446-449; and Shaw et al., 1988, J. Natl. Cancer Inst. 80:1553-1559; Morrison, 1985, Science 229:1202-1207; Oi et al., 1986, Bio/Techniques 4:214; U.S. Pat. No. 5,225,539; Jones et al., 1986, Nature 321:552-525; Verhoeyan et al. (1988) Science 239:1534; and Beidler et al., 1988, J. Immunol. 141:4053-4060; each of which is incorporated herein by reference in its entirety.

Completely human antibodies are particularly desirable for therapeutic treatment of human patients. Such antibodies can be produced using transgenic mice which are incapable of expressing endogenous immunoglobulin heavy and light chains genes, but which can express human heavy and light chain genes. The transgenic mice are immunized in the normal fashion with a selected antigen, e.g., all or a portion of a polypeptide of the invention. Monoclonal antibodies directed against the antigen can be obtained using conventional hybridoma technology. The human immunoglobulin transgenes harbored by the transgenic mice rearrange during B cell differentiation, and subsequently undergo class switching and somatic mutation. Thus, using such a technique, it is possible to produce therapeutically useful IgG, IgA, IgM and IgE antibodies. For an overview of this technology for producing human antibodies, see Lonberg and Huszar (1995, Int. Rev. Immunol. 13:65-93). For a detailed discussion of this technology for producing human antibodies and human monoclonal antibodies and protocols for producing such antibodies, see, e.g., U.S. Pat. No. 5,625,126; U.S. Pat. No. 5,633,425; U.S. Pat. No. 5,569,825; U.S. Pat. No. 5,661,016; and U.S. Pat. No. 5,545,806; each of which is incorporated herein by reference in its entirety. In addition, companies such as Abgenix, Inc. (Freemont, Calif.) and Genpharm (San Jose, Calif.) can be engaged to provide human antibodies directed against a selected antigen using technology similar to that described above.

Completely human antibodies which recognize a selected epitope can be generated using a technique referred to as “guided selection.” In this approach a selected non-human monoclonal antibody, e.g., a mouse antibody, is used to guide the selection of a completely human antibody recognizing the same epitope. (Jespers et al. (1994) Bio/technology 12:899-903).

2. Formulation

An aspect of the invention provides methods of manufacturing a drug useful for treating rheumatoid arthritis in a warm-blooded animal. The drug is prepared in accordance with known formulation techniques to provide a composition suitable for oral, topical, transdermal, rectal, by inhalation, parenteral (intravenous, intramuscular, or intraperitoneal) administration, and the like. Detailed guidance for preparing compositions of the invention are found by reference to the 18^{th or} 19^{th} Edition of Remington's Pharmaceutical. Sciences, Published by the Mack Publishing Co., Easton, Pa. 18040. The pertinent portions are incorporated herein by reference.

Unit doses or multiple dose forms are contemplated, each offering advantages in certain clinical settings. The unit dose would contain a predetermined quantity of an antagonist of CD99 activity calculated to produce the desired effect(s) in the setting of treating rheumatoid arthritis. The multiple dose form may be particularly useful when multiples of single doses, or fractional doses, are required to achieve the desired ends. Either of these dosing forms may have specifications that are dictated by or directly dependent upon the unique characteristic of the particular compound, the particular therapeutic effect to be achieved, and any limitations inherent in the art of preparing the particular compound for treatment of cancer.

A unit dose will contain a therapeutically effectiveamount sufficient to treat rheumatoid arthritis in a subject and may contain from about 1.0 to 1000 mg of compound, for example about 50 to 500 mg.

In a preferred embodiment, the drug of the invention is formulated in accordance with routine procedures as a pharmaceutical composition adapted for intravenous administration to human beings. Typically, pharmaceutical compositions for intravenous administration are solutions in sterile isotonic aqueous buffer. Where necessary, the pharmaceutical composition may also include a solubilizing agent and a local anesthetic such as lignocaine to ease pain at the site of the injection. Generally, the ingredients are supplied either separately or mixed together in unit dosage form, for example, as a dry lyophilized powder or water free concentrate in a hermetically sealed container such as an ampoule or sachette indicating the quantity of active agent. Where the composition is to be administered by infusion, it can be dispensed with an infusion bottle containing sterile pharmaceutical grade water or saline. Where the composition is administered by injection, an ampoule of sterile water for injection or saline can be provided so that the ingredients may be mixed prior to administration.

The drug of the invention can be formulated as neutral or salt forms. Pharmaceutically acceptable salts include those formed with anions such as those derived from hydrochloric, phosphoric, acetic, oxalic, tartaric acids, etc., and those formed with cations such as those derived from sodium, potassium, ammonium, calcium, ferric hydroxides, isopropylamine, triethylamine, 2-ethylamino ethanol, histidine, procaine, etc.

The compound will preferably be administered orally in a suitable formulation as an ingestible tablet, a buccal tablet, capsule, caplet, elixir, suspension, syrup, trouche, wafer, lozenge, and the like. Generally, the most straightforward formulation is a tablet or capsule (individually or collectively designated as an “oral dosage unit”). Suitable formulations are prepared in accordance with a standard formulating techniques available that match the characteristics of the compound to the excipients available for formulating an appropriate composition.

The form may deliver a compound rapidly or may be a sustained-release preparation. The compound may be enclosed in a hard or soft capsule, may be compressed into tablets, or may be incorporated with beverages, food or otherwise into the diet. The percentage of the final composition and the preparations may, of course, be varied and may conveniently range between 1 and 90% of the weight of the final form, e.g., tablet. The amount in such therapeutically useful compositions is such that a suitable dosage will be obtained. Preferred compositions according to the current invention are prepared so that an oral dosage unit form contains between about 5.0 to about 50% by weight (% w) in dosage units weighing between 5 and 1000 mg.

The suitable formulation of an oral dosage unit may also contain: a binder, such as gum tragacanth, acacia, corn starch, gelatin; sweetening agents such as lactose or sucrose; disintegrating agents such as corn starch, alginic acid and the like; a lubricant such as magnesium stearate; or flavoring such a peppermint, oil of wintergreen or the like. Various other material may be present as coating or to otherwise modify the physical form of the oral dosage unit. The oral dosage unit may be coated with shellac, a sugar or both. Syrup or elixir may contain the compound, sucrose as a sweetening agent, methyl and propylparabens as a preservative, a dye and flavoring. Any material utilized should be pharmaceutically-acceptable and substantially non-toxic. Details of the types of excipients useful may be found in the nineteenth edition of “Remington: The Science and Practice of Pharmacy,” Mack Printing Company, Easton, Pa. See particularly chapters 91-93 for a fuller discussion.

The drug of the invention may be administered parenterally, e.g., intravenously, intramuscularly, intravenously, subcutaneously, or interperitonieally. The carrier or excipient or excipient mixture can be a solvent or a dispersive medium containing, for example, various polar or non-polar solvents, suitable mixtures thereof, or oils. As used herein “carrier” or “excipient” means a pharmaceutically acceptable carrier or excipient and includes any and all solvents, dispersive agents or media, coating(s), antimicrobial agents, iso/hypo/hypertonic agents, absorption-modifying agents, and the like. The use of such substances and the agents for pharmaceutically active substances is well known in the art. Except insofar as any conventional media or agent is incompatible with the active ingredient, use in therapeutic compositions is contemplated. Moreover, other or supplementary active ingredients can also be incorporated into the final composition.

Solutions of the compound may be prepared in suitable diluents such as water, ethanol, glycerol, liquid polyethylene glycol(s), various oils, and/or mixtures thereof, and others known to those skilled in the art.

The pharmaceutical forms suitable for injectable use include sterile solutions, dispersions, emulsions, and sterile powders. The final form must be stable under conditions of manufacture and storage. Furthermore, the final pharmaceutical form must be protected against contamination and must, therefore, be able to inhibit the growth of microorganisms such as bacteria or fungi. A single intravenous or intraperitoneal dose can be administered. Alternatively, a slow long term infusion or multiple short term daily infusions may be utilized, typically lasting from 1 to 8 days. Alternate day or dosing once every several days may also be utilized.

Sterile, injectable solutions are prepared by incorporating a compound in the required amount into one or more appropriate solvents to which other ingredients, listed above or known to those skilled in the art, may be added as required. Sterile injectable solutions are prepared by incorporating the compound in the required amount in the appropriate solvent with various other ingredients as required. Sterilizing procedures, such as filtration, then follow. Typically, dispersions are made by incorporating the compound into a sterile vehicle which also contains the dispersion medium and the required other ingredients as indicated above. In the case of a sterile powder, the preferred methods include vacuum drying or freeze drying to which any required ingredients are added.

In all cases the final form, as noted, must be sterile and must also be able to pass readily through an injection device such as a hollow needle. The proper viscosity may be achieved and maintained by the proper choice of solvents or excipients. Moreover, the use of molecular or particulate coatings such as lecithin, the proper selection of particle size in dispersions, or the use of materials with surfactant properties may be utilized.

Prevention or inhibition of growth of microorganisms may be achieved through the addition of one or more antimicrobial agents such as chlorobutanol, ascorbic acid, parabens, thermerosal, or the like. It may also be preferable to include agents that alter the tonicity such as sugars or salts.

In a specific embodiment, it may be desirable to administer the compositions of the invention locally to the area in need of treatment; this may be achieved by, for example, and not by way of limitation, local infusion during surgery, topical application, e.g., in conjunction with a wound dressing after surgery, by injection, by means of a catheter, by means of a suppository, or by means of an implant, said implant being of a porous, non-porous, or gelatinous material, including membranes, such as sialastic membranes, or fibers.

In another embodiment, the composition can be delivered in a vesicle, in particular a liposome (see Langer, 1990, Science 249:1527-1533; Treat et al., in Liposomes in the Therapy of Infectious Disease and Cancer, Lopez-Berestein and Fidler (eds.), Liss, New York, pp. 353-365 (1989); Lopez-Berestein, ibid., pp. 317-327; see generally ibid.)

In yet another embodiment, the composition can be delivered in a controlled release, or sustained release system. In one embodiment, a pump may be used (see Langer, supra; Sefton, 1987, CRC Crit. Ref Biomed. Eng. 14:201; Buchwald et al., 1980, Surgery 88:507; Saudek et al., 1989, N. Engl. J. Med. 321:574). In another embodiment, polymeric materials can be used in a controlled release system (see Medical Applications of Controlled Release, Langer and Wise (eds.), CRC Pres., Boca Raton, Fla. (1974); Controlled Drug Bioavailability, Drug Product Design and Performance, Smolen and Ball (eds.), Wiley, New York (1984); Ranger and Peppas, J., 1983, Macromol. Sci. Rev. Macromol. Chem. 23:61; see also Levy et al., 1985, Science 228:190; During et al., 1989, Ann. Neurol. 25:351; Howard et al., 1989, J. Neurosurg. 71:105). In yet another embodiment, a controlled release system can be placed in proximity of the therapeutic target (e.g., the brain, kidney, stomach, pancreas, and lung), thus requiring only a fraction of the systemic dose (see, e.g., Goodson, in Medical Applications of Controlled Release, supra, vol. 2, pp. 115-138 (1984)).

Other controlled release systems are discussed in the review by Langer (1990, Science 249:1527-1533).

In a specific embodiment where the drug of the invention is a nucleic acid encoding a protein, the nucleic acid can be administered in vivo to promote expression of its encoded protein, by constructing it as part of an appropriate nucleic acid expression vector and administering it so that it becomes intracellular, e.g., by use of a retroviral vector (see U.S. Pat. No. 4,980,286), or by direct injection, or by use of microparticle bombardment (e.g., a gene gun; Biolistic, Dupont), or coating with lipids or cell-surface receptors or transfecting agents, or by administering it in linkage to a homeobox-like peptide which is known to enter the nucleus (see e.g., Joliot et al., 1991, Proc. Natl. Acad. Sci. USA 88:1864-1868), etc. Alternatively, a nucleic acid can be introduced intracellularly and incorporated within host cell DNA for expression, by homologous recombination.

### IV. EXAMPLES

The following examples are provided as a guide for a practitioner of ordinary skill in the art. The examples should not be construed as limiting the invention, as the examples merely provide specific methodology useful in understanding and practicing an embodiment of the invention.

A. Example 1

### Monocyte/T-Cell Recruitment

Cells.

Human PBLs are isolated from the citrate-anticoagulated whole blood of healthy donors or patients with rheumatoid arthritis by dextran sedimentation and density separation over Ficoll-Hypaque. The mononuclear cells are washed and further purified on nylon wool and by plastic adherence, as previously described (Carr 1996). The resulting PBLs (>90% CD3⁺ T lymphocytes) are cultured in LPS-free RPMI/10% FCS for 15-18 h before use. Memory and naive CD3⁺ T lymphocyte subsets (CD45RO⁺ and CD45RA⁺, respectively) are isolated by negative selection using magnetic cell separation (MACS, Miltenyi Biotec, Bergisch Gladbach, Germany), following the manufacturer's instructions. HUVECs are isolated from umbilical cord veins (jaffe 1973) and established as primary cultures in M199 containing 10% FCS, 8% pooled human serum, 50 μg/ml endothelial cell growth factor (Sigma-Aldrich), 10 U/ml porcine intestinal heparin (Sigma-Aldrich), and antibiotics. Experiments are done on cells at passage two cultured on hydrated Type I collagen gels (Muller 1989) in 96-well culture plates. In certain experiments TNF-α or IL-1^{β} (10 ng/ml and 10 U/ml final concentrations, respectively) or diluted synovial fluid from healthy donors or patients with rheumatoid arthritis are added to the culture media for the final 4-24 h.

Antibodies.

Antibodies to CD18 (IB4), CD14 (3C10) and MHC class II (9.3C9) from the American Type Culture Collection (Rockville, Md.) are used as negative control. Anti-CD31 is used as a positive control from transendothelial migration blockade. Anti-CD3 (OKT-3) and anti-CD28 (leu28) mAbs are used in the T cell activation assays.

The migration of monocytes or T-cells through a layer of endothelial cells is measured. The details of this assay are described in Muller et al., J Exp Med 176:819-828 (1992) and Muller et al., J Exp Med 178:449-460 (1993). Transendothelial migration is quantitated by Namarski optics as described in Liao et al., J Exp Med 182:1337-1343 (1995) and Muller et al., J Exp Med 178:449-460 (1993). Leukocytes are isolated from the peripheral blood of healthy volunteers or patients with rheumatoid arthritis and added to confluent monolayers of HUVECs grown on hydrated collagen gels previously incubated with anti-CD99 mAb, 12E7. After incubation (1 h unless otherwise reported), nonadherent cells are removed by washing and the remaining adherent and transmigrated cells are fixed in place on the endothelial monolayer by overnight incubation in 2.5% glutaraldehyde in 0.1 M sodium cacodylate buffer at pH 7.4. Multiple high-power fields are observed under microscope and scored. Transmigration data are expressed as the percentage of the total cells that remained with the monolayer that were below the endothelium. In certain experiments, anti-CD99 or control mAbs are incubated with leukocytes, endothelial cells or both for 30 min before the assay, and then removed by extensive washing before the start of transmigration. In certain experiments, at the completion of the standard transmigration time, blocking mAbs were washed away and the cultures returned to the incubator in complete medium with or without blocking concentrations of the same or an alternate mAb.

Transendothelial migration is also quantitated on cross-sections of paraffin-embedded monolayers. These specimens are prepared by carefully removing replicate sample monolayers and placing the endothelial surfaces against each other with the collagen gel sides facing outward. This avoids mechanical dislodgement of cells during the embedding process. After substitution in wax, the specimens are bisected so that cuts through the specimen produce cross sections of four monolayer samples (two different portions of each of the two monolayers). Quantitation is performed on three levels of such specimens separated y at least 50 μm so that different areas of the specimen are sampled and the same cells are not counted twice.

### B. Example 2

### T-Cell Proliferation and Activation T Cell Proliferation Assays

Proliferation assays of highly purified PB T cells derived from healthy volunteers or patients with rheumatoid arthritis (5×104 cells/well) are performed in triplicate in 96-well U-bottom tissue culture plates in a final volume of 200 μl. Proliferation is induced by the anti-CD3 mAbs+anti-CD99 or control mAbs (5 μg/ml) cross-linked with GAM-IgG (10 μg/ml; Sigma) and by PMA (Sigma; final concentration, 10-7 M) or ionomycin (Sigma; final concentration, 1 μM). For proliferation experiments with immobilized CD3 mAb, 96-well flat-bottom plates (Costar) are coated overnight at 4° C. with 100 μl of 0.125 to 1.0 μg/ml of purified OKT3 mAb diluted in PBS. The plates are washed twice with PBS and subsequently used for the assays. PMA (Sigma), ionomycin (Sigma), and the mAbs are diluted in RPMI 1640 supplemented with 10% FCS, 2 mM L-glutamine, 10 U/ml penicillin, and 100 μg/ml streptomycin. GAM-IgG and the cells are resuspended in RPMI 1640 supplemented with 10% pooled human serum.

After 72 h of incubation in a humidified atmosphere with 5% CO₂ at 37° C., the cells are pulsed with 1 μCi/well of [methyl-3H]TdR (Amersham). Eighteen hours later the cell lysates are harvested on glass-fiber filters and radioactivity was determined on a microplate scintillation counter.

Determination of Intracellular Cytokines

For generation of PHAIIL-2-dependent blasts, PBMC (1×105/well) are cultured in RPMI 1640 plus 10% FCS (Life Technologies) supplemented with antibiotics in the presence of PHA (Sigma; final concentration, 1 μg/ml) in 96-well U-bottom culture plates (Costar) for 7 days. Subsequently, every 5 to 7 days 10 U/ml of IL-2 plus autologous irradiated (3000 rad, 137Cs source) PBMC as feeder cells (ratio of blasts/feeder cells=1:1) are added. The cells are cultured for at least 1 mo before the first experiments are performed.

Ninety-six-well flat-bottom tissue culture plates (Costar) are coated with GAM-IgG (Sigma; 10 μg/ml) plus a suboptimal concentration of the CD3 mAb OKT3 (20 ng/ml) at 4° C. overnight. After two washings with PBS, T cell lines or clones (1-2×105/well) are incubated in precoated plates with optimal concentrations (5 μg/ml) of CD99 mAbs, CD28 mAb Leu28, or isotype control mAb. Assays are set up in a total volume of 200 μl/well in RPMI 1640 medium containing 5% pooled human serum supplemented with antibiotics and 2 μg/ml (final concentration) of brefeldin A (Sigma). After 18 h of incubation at 37° C. in a 5% CO₂ atmosphere, the cells were harvested and analyzed for the presence of intracellular cytokines. For staining, 50 μl of the cell suspension (corresponding to 1-2×105 cells) are fixed for 30 min at room temperature by the addition of 100 μl of 1% paraformaldehyde. Subsequently, cells were washed once with 4 ml of PBS/1% BSA, resuspended in 50 μl of PBS/1% BSA, permeabilized by the addition of 100 μl of PERM solution (BD Pharmingen), and incubated for 30 min at room temperature with the indicated directly conjugated anti-cytokine mAb. Finally, cells were washed twice, resuspended in PBS, and analyzed by flow cytometry.

For quantitative measurement of secreted cytokines, 1×10⁶ T cells are incubated on anti-CD3 coated plates with the anti-CD99 or control mAbs for 72 h at 37° C./5% CO₂. Culture supernatants were then harvested and a sandwich ELISA assay is performed to measure the production of IL-4, IL-10, TNF-α and IFN-γ using manufacturer's protocol (R&D Systems Inc., Minneapolis, Minn.) Control experiment: Stimulate T-cells and measure production of interferon gamma

All publications and patents mentioned in the above specification are herein incorporated by reference. Various modifications and variations of the described method and system of the invention will be apparent to those skilled in the art without departing from the scope and spirit of the invention. Although the invention has been described in connection with specific preferred embodiments, it should be understood that the invention as claimed should not be unduly-limited to such specific embodiments. Indeed, various modifications of the described modes for carrying out the invention which are obvious to those skilled in the art are intended to be within the scope of the following claims.

1. A method of alleviating at least one symptom of rheumatoid arthritis comprising administering a therapeutically effective amount of an antagonist of CD99 activity to a patient having rheumatoid arthritis.

2. The method of claim 1, wherein the antagonist of CD99 activity is a protein.

3. The method of claim 2, wherein the protein is an anti-CD99 antibody.

4. The method of claim 3, wherein the antibody is a monoclonal antibody.

5. The method of claim 4, wherein the anti-CD99 monoclonal antibody is selected from the group consisting of Hec2, D44, O662, MEM-131, TU12, HO36-1.1, HIT4, O13, N-16, C-20, B-N24, 12E7, 3B2/TA8, HI142, HI175, FMC29, HI147, HI170, L129, and Ad20.

6. The method of claim 5, wherein the anti-CD99 monoclonal antibody is selected from the group consisting of Hec2, D44 and O662.

7. The method of claim 1, wherein the antagonist of CD99 activity is a nucleic acid.

8. The method of claim 7, wherein the nucleic acid is an antisense inhibitor.

9. The method of claim 8, wherein the antisense inhibitor comprises a portion of the mic2 gene transcribed in reverse orientation.

10. The method of claim 1, wherein the antagonist of CD99 activity is a small molecule.

11. The method of claim 1, wherein the patient is a methotrexate resistant patient.

12. The method of claim 1, wherein the patient is a TNF-α blockade cartilage nonresponder.

13. The method of claim 1, wherein the patient is a TNF-α blockade hyperplasia nonresponder.

14. The method of claim 1, wherein the patient is a TNF-α blockade double nonresponder.

15-63. (canceled)

64. A method for identifying a compound useful for treatment of rheumatoid arthritis, comprising:
(a) comparing an amount of leukocytes that migrate through at least one layer of endothelial cells in the presence of the compound with an amount of leukocytes that migrate through at least one layer of endothelial cells in the absence of the compound; and
(b) identifying the compound as useful for treatment of rheumatoid arthritis when the amount of migrating leukocytes in the presence of the compound is less than the amount of migrating leukocytes in the absence of the compound.

65. The method of claim 64, wherein the endothelial cells are cultured human umbilical vein endothelial cells.

66. The method of claim 64, wherein the endothelial cells are stimulated with tumor necrosis factor or interleukin-1.

67. The method of claim 64, wherein the at least one layer of endothelial cells is a monolayer of endothelial cells.

68. The method of claim 64, wherein the leukocytes are monocytes.

69. The method of claim 64, wherein the leukocytes are T-cells.

70. A method of manufacturing a drug for use in the treatment of rheumatoid arthritis comprising:
(a) identifying a compound as an antagonist of CD99 activity by:
(i) comparing an amount of leukocytes that migrate through at least one layer of endothelial cells in the presence of the compound with an amount of leukocytes that migrate through at least one layer of endothelial cells in the absence of the compound; and
(ii) identifying a compound as useful in the treatment of rheumatoid arthritis when the amount of migrating leukocytes in the presence of the compound is less than the amount of migrating leukocytes in the absence of the compound; and
(b) formulating said compound for human consumption.

71. A method for identifying a compound useful for treatment of rheumatoid arthritis, comprising:
(a) comparing an amount of a T-cell cytokine produced by a first population of T-cells activated with a non-blocking anti-CD99 antibody in the presence of the compound with an amount of the T-cell cytokine produced by a second population of T-cells activated with a non-blocking anti-CD99 antibody in the absence of the compound; and
(b) identifying the compound useful for treatment of rheumatoid arthritis when the amount of the T-cell cytokine produced in the presence of the compound is less than the amount of the T-cell cytokine produced in the absence of the compound.

72. The method of claim 71, wherein the non-blocking anti-CD99 antibody is 12E7 or 3B2/TA8.

73. The method of claim 71, wherein the T-cell cytokine is a Th1 cytokine.

74. The method of claim 73, wherein the Th1 cytokine is TNF-α or IFNγ.

75. The method of claim 71, wherein the T-cells are Th1 T-cells.

76. A method of manufacturing a drug for use in the treatment of rheumatoid arthritis comprising:
(a) identifying a compound as an antagonist of CD99 activity by:
(i) comparing an amount of a T-cell cytokine produced by a first population of T-cells activated with a non-blocking anti-CD99 antibody in the presence of the compound with an amount of the T-cell cytokine produced by a second population of T-cells activated with a non-blocking anti-CD99 antibody in the absence of the compound; and
(ii) identifying the compound as an antagonist of CD99 activity when the amount of the T-cell cytokine produced in the presence of the compound is less than the amount of the T-cell cytokine produced in the absence of the compound; and
(b) formulating said compound for human consumption.

77. A method of identifying a compound useful for treatment of rheumatoid arthritis comprising:
(a) comparing a number of a T-cells developed from a first population of T-cells-stimulated with a non-blocking anti-CD99 antibody in the presence of the compound with a number of T-cells developed from a second population of T-cells in the absence of the compound, wherein the number of T-cells in the first population and second population are the same prior to stimulation with the non-blocking anti-CD99 antibody and exposure to the compound; and
(b) identifying the compound as useful for treatment of rheumatoid arthritis when the number of T-cells developed in the presence of the compound is less than the number of T-cells developed in the absence of the compound.

78. The method of claim 77, wherein the T-cells are Th1 T-cells.

79. A method of manufacturing a drug for use in the treatment of rheumatoid arthritis comprising:
(a) identifying a compound as an antagonist of CD99 activity by:
(i) comparing a number of a T-cells developed from a first population of T-cells stimulated with a non-blocking anti-CD99 antibody in the presence of the compound with a number of T-cells developed from a second population of T-cells in the absence of the compound, wherein the number of T-cells in the first population and second population are the same prior to stimulation with the non-blocking anti-CD99 antibody and exposure to the compound; and
(ii) identifying the compound as an antagonist of CD99 activity when the number of T-cells developed in the presence of the compound is less than the number of T-cells developed in the absence of the compound; and
(b) formulating said compound for human consumption.

80. A method of screening a collection of compounds for use in the treatment of rheumatoid arthritis comprising:
(a) comparing an amount of leukocytes that migrate through at least one layer of endothelial cells in the presence of a compound of the collection with an amount of leukocytes that migrate through at least one layer of endothelial cells in the absence of the compound; and
(b) selecting the compound as useful for the treatment of rheumatoid arthritis when the amount of migrating leukocytes in the presence of the compound is less than the amount of migrating leukocytes in the absence of the compound.

81. The method of claim 80 further comprising repeating steps (a) and (b) for each compound of the collection, wherein at least one compound of the collection is selected as useful for the treatment of rheumatoid arthritis.

82-86. (canceled)

87. A method of screening a collection of compounds for CD99 antagonistic properties comprising:
(a) comparing an amount of a T-cell cytokine produced by a first population of T-cells activated with a non-blocking anti-CD99 antibody in the presence of the compound with an amount of the T-cell cytokine produced by a second population of T-cells activated with a non-blocking anti-CD99 antibody in the absence of the compound; and
(b) selecting the compound as an antagonist of CD99 activity when the amount of the T-cell cytokine produced in the presence of the compound is less than the amount of the T-cell cytokine produced in the absence of the compound.

88. The method of claim 87 further comprising repeating steps (a) and (b) for each compound of the collection, wherein at least one compound of the collection is selected as an antagonist of CD99 activity.

89-92. (canceled)

93. A method of screening a collection of compounds for use in the treatment of rheumatoid arthritis comprising:
(a) comparing an amount of a T-cell cytokine produced by a first population of T-cells activated with a non-blocking anti-CD99 antibody in the presence of the compound with an amount of the T-cell cytokine produced by a second population of T-cells activated with a non-blocking anti-CD99 antibody in the absence of the compound; and
(b) selecting the compound as useful in treating rheumatoid arthritis when the amount of the T-cell cytokine produced in the presence of the compound is less than the amount of the T-cell cytokine produced in the absence of the compound.

94. The method of claim 93 further comprising repeating steps (a) and (b) for each compound of the collection, wherein at least one compound of the collection is selected as useful in treating rheumatoid arthritis.

95-98. (canceled)

99. A method of screening a collection of compounds for use in treatment of rheumatoid arthritis comprising:
(a) comparing a number of T-cells developed from a first population of T-cells stimulated with a non-blocking anti-CD99 antibody in the presence of the compound with a number of T-cells developed from a second population of T-cells in the absence of the compound, wherein the number of T-cells in the first population and the second population are the same prior to stimulation with the non-blocking anti-CD99 antibody and exposure to the compound; and
(b) selecting the compound for use in treatment of rheumatoid arthritis when the number of T-cells developed in the presence of the compound is less than the number of T-cells developed in the absence of the compound.

100. The method of claim 99 further comprising repeating steps (a) and (b) for each compound of the collection, wherein at least one compound of the collection is selected as useful in treating rheumatoid arthritis.

101-105. (canceled)

106. The method of claim 1, wherein the symptom of rheumatoid arthritis is selected from the group consisting of an abnormally increased synovial cell density, an abnormally high rate of cartilage degradation, an abnormally high concentration of IL-6 in synovial tissue and an abnormally high rate of bone erosion.
